(19) United States
(12) Patent Application Publication
(10) Pub. No.: US 2006/0095006 A1
(43) Pub. Date: May 4, 2006

(54) Screw driver structure

(76) Inventors: Chang-Ming Yang, Miaoli (TW); Chun-Mei Chou, Miaoli (TW)

Correspondence Address: APEX JURIS, PLLC;TRACY M HEIMS, LAKE CITY CENTER, SUITE 410, 12360 LAKE CITY WAY NORTHEAST, SEATTLE, WA, 98125, US

(21) Appl. No.: 10/976,462

(22) Filed: Oct. 29, 2004

(51) Int. Cl. A61M 5/00

(52) U.S. Cl. 604/110

(57) ABSTRACT

A one-time safe administration set structure comprises essentially a protective rider, a gliding part, and a hose, wherein said protective rider is provided thereon with a slideway, said slideway is provided on its rear side with a receptive portion, and said gliding part is engaged with a hand pushing part through a sliding block on the upper edge of said gliding part. In use of said one-time safe administration set structure, said hand pushing part is pushed forward such that the rear end of said gliding part is stopped by the protruding button provided near the front end of said slideway, thereby actions of injection or drawing can be carried out. After its use, said hand pushing part is pushed backward, passes through the stopping block provided between the slideway and the receptive portion, and then is pushed down into the receptive portion, whereby said stopping block can withstand against the upper edge of said hand pushing part such that it is fixed and hence is safe without worrying about its recycled use.

### BACKGROUND OF THE INVENTION

1. Field of the invention

The invention relates to a one-time safe administration set structure, and in particular, to a safe, easy operable, one-time, safe administration set structure without worrying about its recycled use.

2. Description of the Prior art

As is known, a conventional administration set is composed of a syringe and a needle cover, wherein, said syringe is provided laterally on the main body several sidepieces, and said main body is inserted at its front end with an injection needle, while said needle cover is in cylindrical form and has a receptive port provided on its bottom. In maintenance and use of this conventional administration set, the needle cover should be removed at first from the syringe, and then the injection needle is inserted into the injection site of a subject. However, after using this conventional administration set, the needle cover must cover again the syringe having an injection needle still provided thereon, such that the medical practitioner might be susceptive to be hurt and hence infected.

Accordingly, the above-mentioned administration set has still many disadvantages, not a perfect design and need to be improved.

In view of above-mentioned disadvantages derived from the prior art, the inventor of this application had thought to improve it and, after studying intensively for many years, has developed successfully the one-time safe administration set according to the invention.

### SUMMARY OF THE INVENTION

Accordingly, one object of the invention is to provide a safe, easy operable one-time safe administration set.

Another object of the invention is to provide a one-time safe administration set without worrying secondary use.

The one-time safe administration set structure that can achieve the above-described objects of the invention comprises:

a protective rider, provided thereon with a slideway, wherein said slideway is provided on its rear side with a receptive portion, and with protruding buttons near its front and rear ends, and wherein said protective rider is provided with several stopping blocks at junction between said slideway and said receptive portion as well as around said receptive portion;

a gliding part, being engaged with a hand pushing part through a sliding block on the upper edge of said gliding part, wherein said gliding part is set in said protective rider and is fixed at a place between said protruding button provided at the rear end of said slideway and said stopping block; and

a hose, provided at its end with an adapting head, which can connect with a usual syringe for injection or drawing or catheter for infusion.

These features and advantages of the present invention will be fully understood and appreciated from the following detailed description of the accompanying Drawings.

### BRIEF DESCRIPTION OF THE DRAWINGS

The drawings disclose an illustrative embodiment of the present invention which serves to exemplify the various advantages and objects hereof, and are as follows:

FIG. 1 is a three-dimensional exploded view of the one-time safe administration set structure;

FIG. 1A is a three-dimensional exploded view of a one-time administration set structure according to a first embodiment of the invention;

FIG. 1B is a three-dimensional exploded view of a one-time administration set structure according to a second embodiment of the invention;

FIG. 1C is a three-dimensional exploded view of a one-time administration set structure according to a third embodiment of the invention;

FIG. 1D is a three-dimensional exploded view of a one-time administration set structure according to a fourth embodiment of the invention;

FIG. 1E is a exploded side view of a one-time administration set structure according to a fifth embodiment of the invention;

FIG. 1F is a three-dimensional exploded view of a one-time administration set structure according to a sixth embodiment of the invention;

FIG. 2 is a three-dimensional exploded view of a one-time administration set structure according to a seventh embodiment of the invention;

FIG. 3 is a three-dimensional exploded view of a one-time administration set structure according to an eighth embodiment of the invention;

FIG. 4 is a three-dimensional exploded view of a one-time administration set structure according to a ninth embodiment of the invention;

FIG. 5 is a three-dimensional aspect view of a one-time administration set structure according to a tenth embodiment of the invention;

FIG. 5A is a three-dimensional aspect view of a one-time administration set structure according to a eleventh embodiment of the invention;

FIG. 5B is a three-dimensional view of a one-time administration set structure according to a twelfth embodiment of the invention;

FIG. 6 is a three-dimensional view of a one-time administration set structure according to a 13th embodiment of the invention;

FIG. 7 is a schematic view showing the action of a one-time administration set structure according to a 14th embodiment of the invention;

FIG. 8 is a schematic view showing the action of a one-time administration set structure according to a 15th embodiment of the invention;

FIG. 9 is a three-dimensional aspect view of a one-time administration set structure according to a 16th embodiment of the invention;

FIG. 10 is a three-dimensional view of a one-time administration set structure according to a 17th embodiment of the invention;

FIG. 11 is a three-dimensional aspect view of a one-time administration set structure according to an 18th embodiment of the invention;

FIG. 11A is a three-dimensional aspect view of a one-time administration set structure according to a 19th embodiment of the invention;

FIG. 11B is a three-dimensional aspect view of a one-time administration set structure according to a 20th embodiment of the invention;

FIG. 12 is a three-dimensional aspect view of a one-time administration set structure according to a 21st embodiment of the invention;

FIG. 13 is a three-dimensional aspect view of a one-time administration set structure according to a 22nd embodiment of the invention;

FIG. 13A is a three-dimensional aspect view of a one-time administration set structure according to a 23rd embodiment of the invention;

FIG. 14 is a three-dimensional exploded view of a one-time administration set structure according to a 24th embodiment of the invention;

FIG. 14A is a three-dimensional aspect view of a one-time administration set structure according to a 25th embodiment of the invention;

FIG. 14B is a three-dimensional aspect view of a one-time administration set structure according to a 26th embodiment of the invention;

FIG. 14C is a three-dimensional aspect view of a one-time administration set structure according to a 27th embodiment of the invention;

FIG. 14D is a three-dimensional aspect view of a one-time administration set structure according to a 28th embodiment of the invention;

FIG. 14E is a three-dimensional aspect view of a one-time administration set structure according to a 29th embodiment of the invention;

FIG. 15 is a three-dimensional exploded view of the gliding part in a one-time administration set structure according to another embodiment of the invention;

FIG. 16 is a three-dimensional exploded view of a one-time administration set structure according to a 30th embodiment of the invention;

FIG. 17 is a three-dimensional exploded view of a one-time administration set structure according to a 31st embodiment of the invention;

FIG. 18 is a three-dimensional exploded view of a one-time administration set structure according to a 32nd embodiment of the invention;

FIG. 19 is a three-dimensional exploded view of a one-time administration set structure according to a 33rd embodiment of the invention;

FIG. 20 is a three-dimensional exploded view of a one-time administration set structure according to a 34th embodiment of the invention; and

FIG. 20A is a three-dimensional exploded view of the main body of a one-time administration set structure according to another embodiment of the invention.

### DETAILED DESCRIPTION OF THE PREFERRED EMBODIMENT

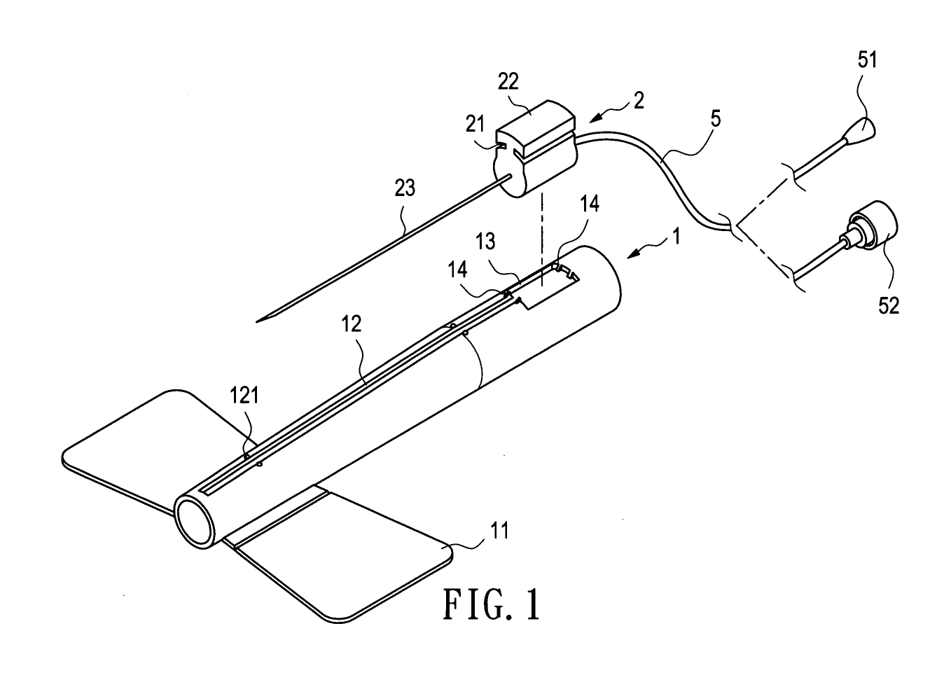

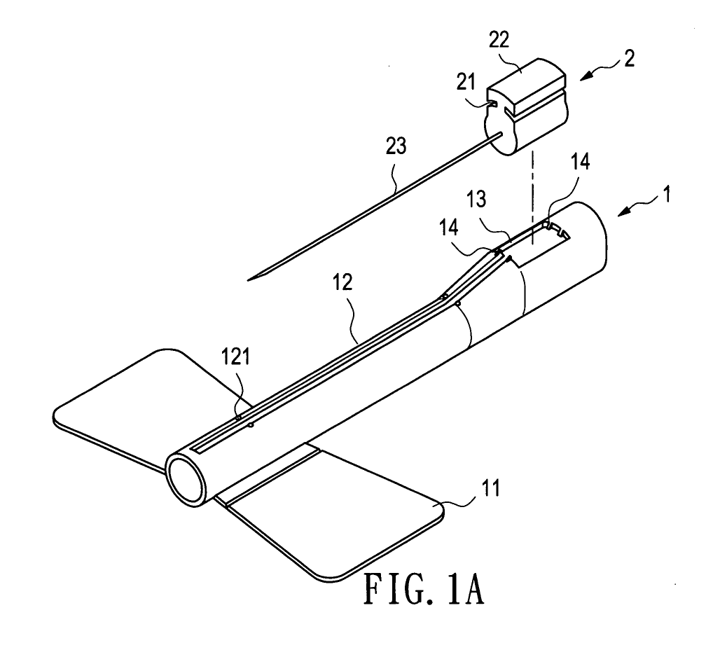

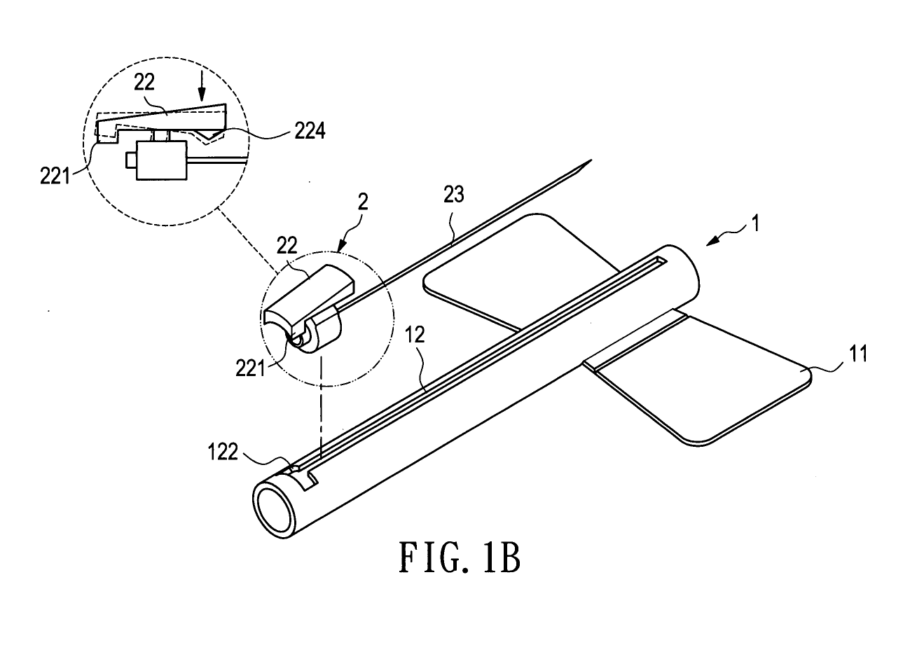

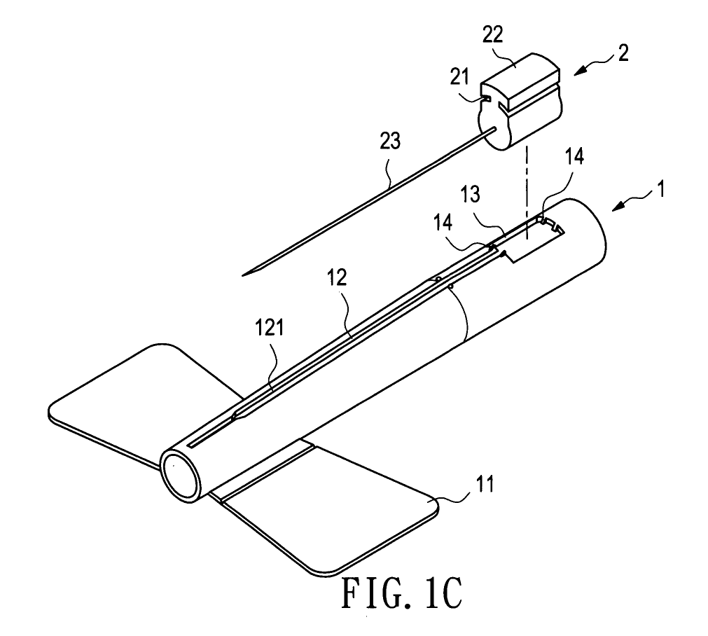

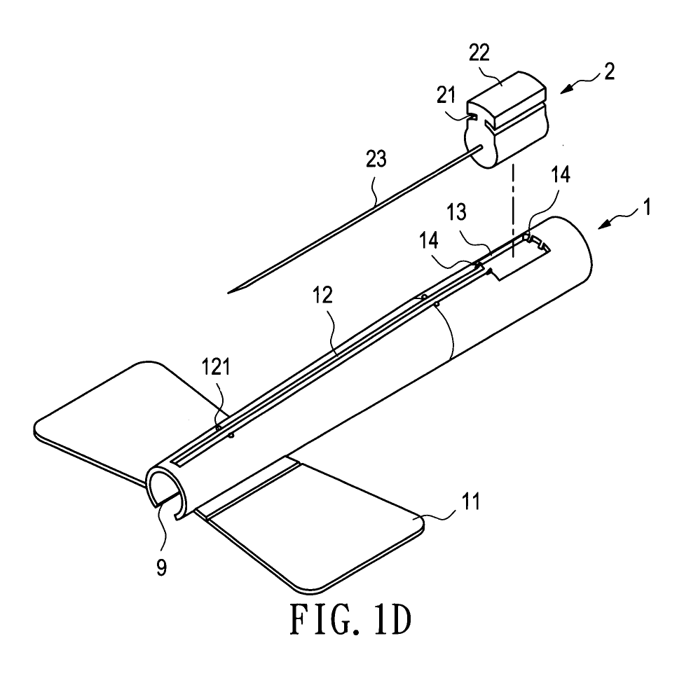

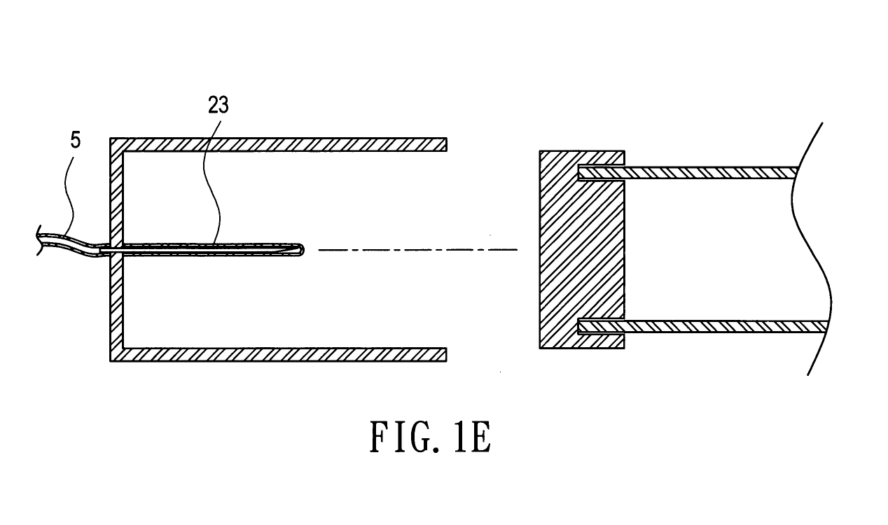

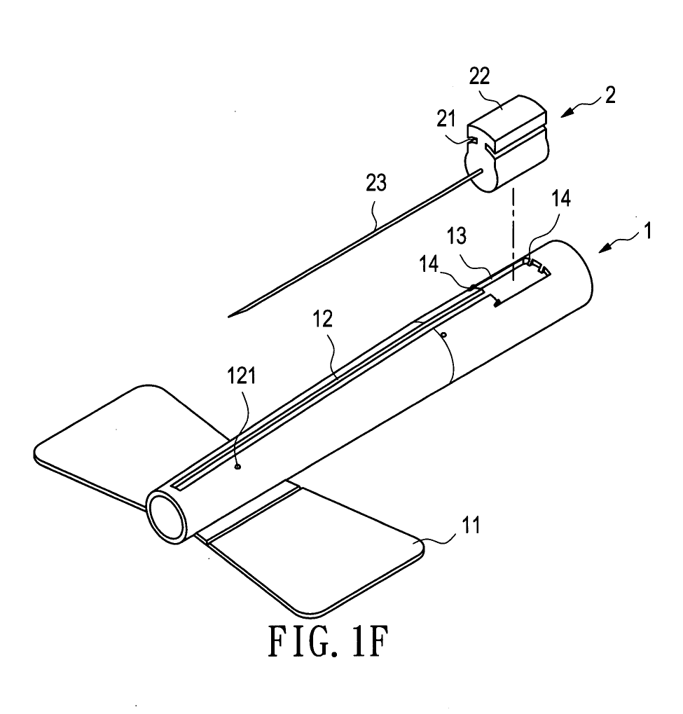

Now referring to FIG. 1, 1A and 1E, the one-time administration set provided by the invention comprises:

a protective rider 1, provided thereon with one or two butterfly slice 11 extended laterally from the front bottom of said protective rider 1, and also provided with a slideway 12, wherein said slideway 12 is provided on its rear side with a receptive portion 13, and with protruding buttons 121 near its front and rear ends, and wherein said protective rider 1 is provided with several stopping blocks 14 at junction between said slideway 12 and said receptive portion 13 as well as around said receptive portion 13; and further, said protective rider 1 is tapered forwardly from the protruding button 121 at its rear end;

a gliding part 2, engaged with a hand pushing part 22 through a sliding block 22 on the upper edge of said gliding part;

a hose 5, provided at its end with an adapting head 51 which can connect with a usual syringe for injection or drawing or catheter for infusion, and which, alternatively, can be replaced with a screw adapting head 52 that can connect with a special syringe having screw thread;

and furthermore, at the rear end of said hose 5, it being possible to connect with a syringe which can be provided with an injection needle 23 and wherein an external annular jacket 27is provided around the periphery of said injection needle 23.

Said gliding part 1 is set by inserting in said protective rider 1, and is fixed at a place between said protruding button 121 at the rear end of said slideway 12 and said stopping block 14. In the use of said one-time safe administration set structure, the hand pushing part 22 is pushed forward that brings the gliding part 2 to move forward, detach from the protruding button 121 provided near the rear end of the slideway 12, and reach at the front end of the slideway 12 such that the gliding part 2 is pushed over the protruding button 121 provided near the rear end of the slideway 12 where the rear end of the gliding part 2 is resisted by the protruding button 121 provided near the rear end of the slideway 12 and hence can not move back, then actions of injection or drawing can be carried out at this situation. After the injection or drawing is complete, the one-time administration set structure according to the invention can be workup just by pushing the hand pushing part 22 backward to pass over the stopping block 14 positioned between the slideway 12 and the receptive portion 13, and then pushing the hand pushing part 22 downward into the receptive portion 13, where the stopping block 14 provided around the receptive portion 13 can resist against the upper edge of the hand pushing part 22 such that two protection devices can be provided by using the periphery of the receptive portion 13 and the stopping block 14 at the junction between the slideway 12 and the receptive portion 13 for the one-time safe administration set according to the invention from secondary use. Moreover, instead of being tapered from the protruding button 121 provided at the rear end of the slideway 12 forwardly, said protective rider 1 could be raised upwardly from the protruding button 121 provided at the rear end of the slideway 12. Further, referring to FIG. 1F, the protruding button 121 on the protective rider 1 and the stopping block 14 provided at the junction between the slideway 12 and the receptive portion 13 can alternatively provided around the outside periphery of the protective rider 1, and can be at the junction with the bottom of the hand pushing part 22.

Referring to FIG. 1B, in one embodiment, the protective rider 1 in the one-time safe administration set structure may not be necessarily provided with the stopping block 14 as well as the protruding button 121. Further, said protective rider 1 may not take a form of being tapered from the protruding button 121 provided at the rear end of the slideway 12 forwardly. Instead, a protruding block 221 is provided at the end tail part of the hand pushing part 22 of the gliding part 2 in a manner that, as the gliding part 2 engages with the protective rider 1, said protruding block 221 can contact with the outside wall surface of the protective rider 1 to generate a frictional force and hence hinder the gliding part 2 from readily displacing. In this embodiment, when the one-time safe administration set according to the invention is used, it needs simply press the front end of the hand pushing part 22 to let the protruding block 221 hike by using the gliding block 21 as the axis such that there is a gap generated between the protruding block 221 and the outside wall surface of the protective rider 1, thereby, the gliding part 2 can be pushed back and forth by means of the hand pushing part 22. As the one-time safe administration set structure according to the invention is used, it needs simply to push the gliding part 2 to the end of the slideway 121 to let the protruding block 221 falling into the receptive portion 12, whereby, the protruding block 221 can not release from the receptive portion 13 even as the hand pushing part 22 is pressed, and hence the purpose of preventing from secondary use is achieved; further, in one embodiment of the one-time administration set according to the invention protruding portion 224 is also provided at the bottom of the front end of the hand pushing part 22, and a slope is provided over the front and rear ends of the protruding portion 224, such that, as the gliding part 2 is pushed to the front end of the protective rider 1, said protruding portion 224 will detach off the slideway 12 by means of its slope over its front end and move to the front edge of the protective rider. Under these circumstance, as the injection or drawing is carried out, said protective rider 2 could be resisted against the front wall surface by means of the protruding portion 224 so as to prevent the receding of the injection needle 23. When the drawing or injection is complete, by simply pushing the gliding part backward by means of the hand pushing part 22, the protruding portion 224 can detach off the front wall surface of said protective rider 1 by means of the rear slope of the protruding portion 224 and return into the slideway 12.

Referring to FIGS. 1C and 1D, in one embodiment of the one-time safe administration set structure according to the invention, the front end of the slideway 12 can be in an invaginate form, while the slideway 12 is not provided with any protruding block 12 at a position near its front end. Thus, as the gliding part 2 moves to the front end of the slideway 12, the gliding block 21 is forced by the invaginate slideway 12 so that the gliding part 2 is fixed at the front end of the protective rider 1, which facilitates the action of injection or drawing using the injection needle. Further, in the one-time safe administration set according to the invention, the bottom at the front of the protective rider 1 may be provided with a opening 9 to avoid the influence of the outside wall thickness of the protective rider 1 on the injection needle during drawing or injecting, such as, for example, an excessive angle formed against the injection site on the subject resulting an inconvenience on the injection or drawing.

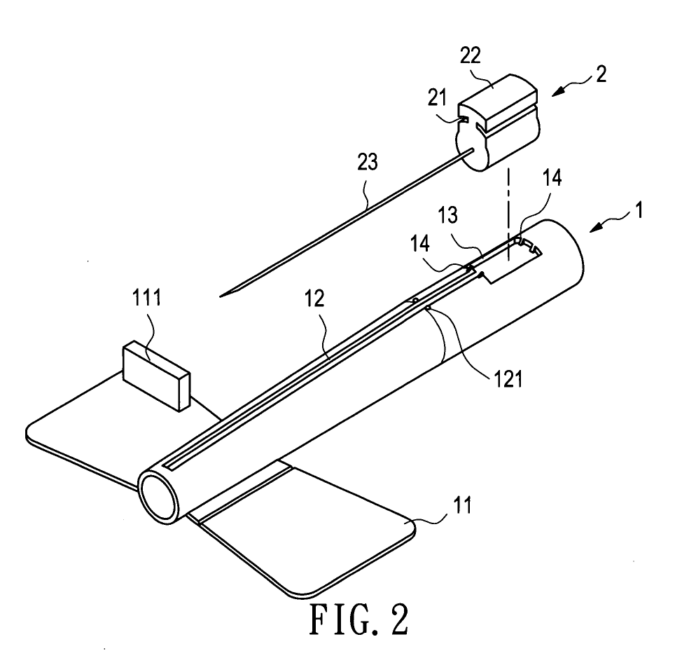

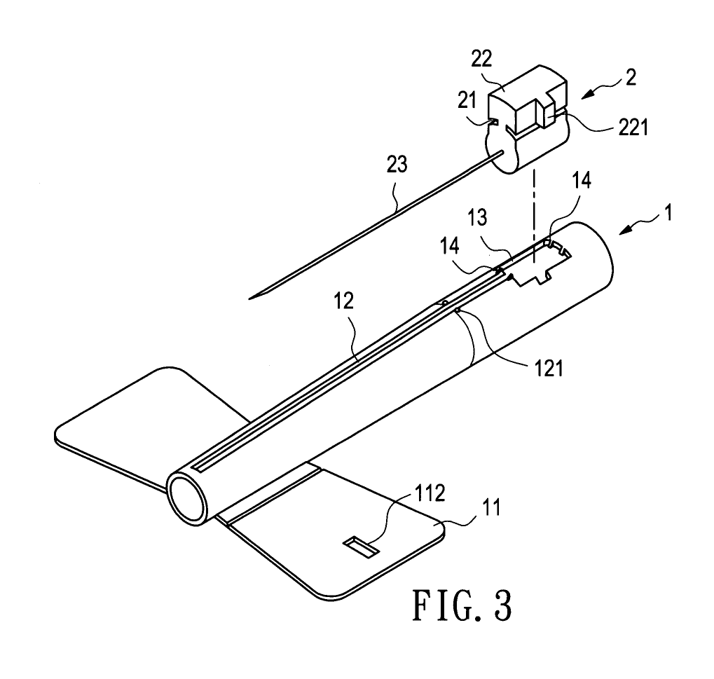

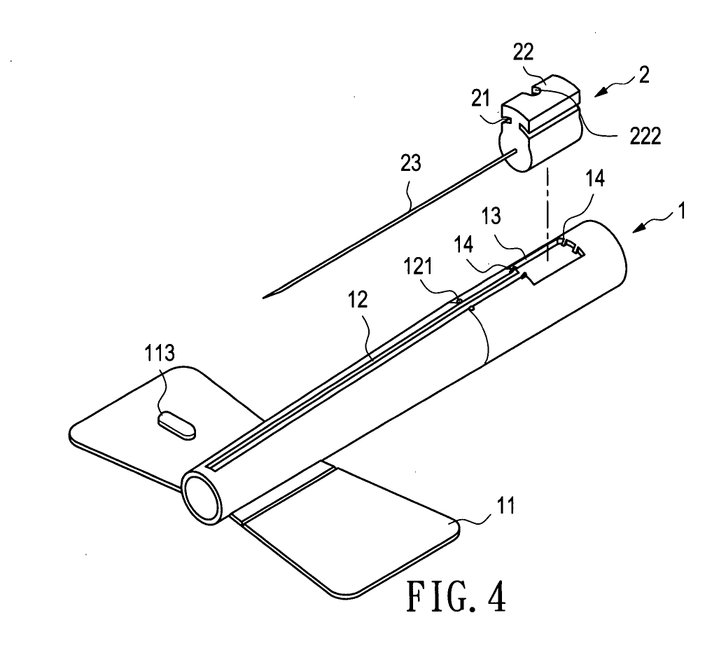

Referring to FIGS. 2, 3 and 4, in the one-time safe administration set structure, a butterfly slice 11 is provided on both sides of the protective rider 1, and no protruding button 121 is provided at the position near the front end of the slideway 12, while a stopping block 111 is provided on the butterfly slice 11 at one or two sides of the protective rider 1. Thus, as the hand pushing part 22 is pushed to the front end of the slideway 12, the stopping block 111 can resist against the rear side of the hand pushing part 22 by bending the butterfly slice 11, thereby preventing the receding of the hand pushing part 22. Under this situation, an injection or drawing can be carried out by inserting the injection needle 23 provided at the front end of the gliding part 2 into the injection site of the subject. Alternatively, a protruding portion 221 can be provided extending outwardly from one or two sides of the hand pushing part 22, respectively, and a grommet 112 for fastening to the corresponding protruding portion 221 can be provided on the butterfly slice 11 of the protective rider 1, or a recess 222 is provided on one or two sides of the hand pushing part 22 and a fastening block 113 is provided on the butterfly slice 11 of the protective rider 1 for fastening into the recess 222. Thus, through the fastening action between the protruding portion 221 and the grommet 112 or between the recess 222 and the fastening block 113, as the hand pushing part 22 is pushed to reach the front end of the slideway 12, the hand pushing part 22 can be prevent from receding by bending the butterfly slice 11 to resist against the rear side of the hand pushing part 22, thereby facilitating the operation of injection or drawing.

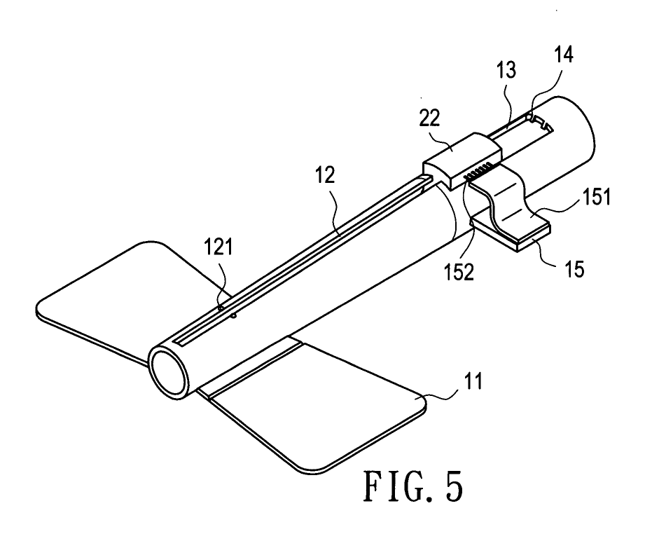

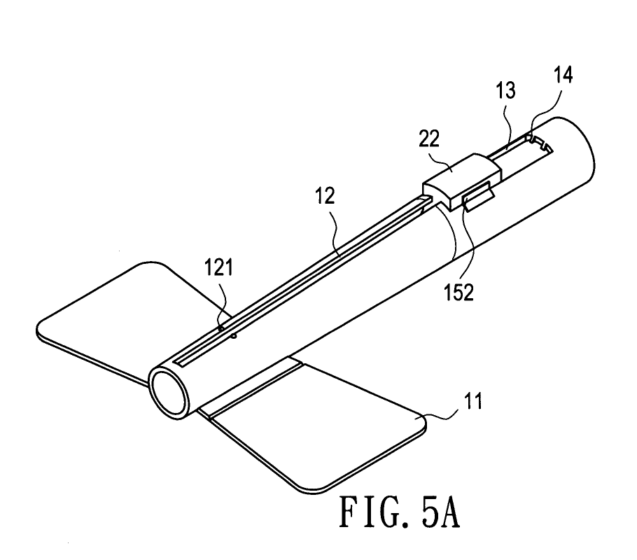

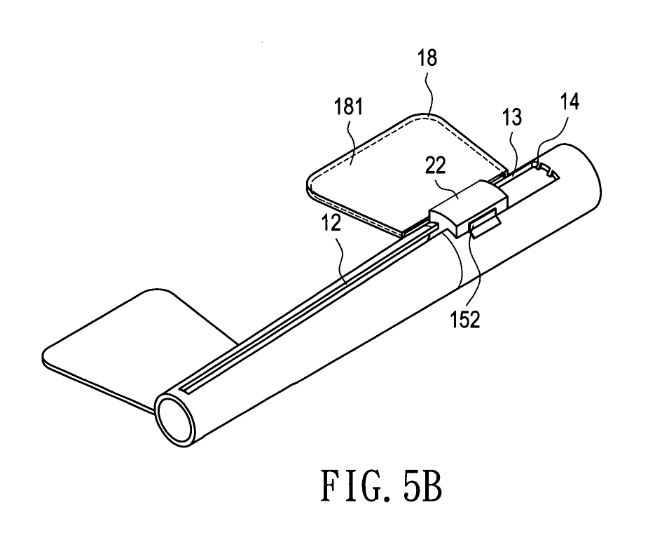

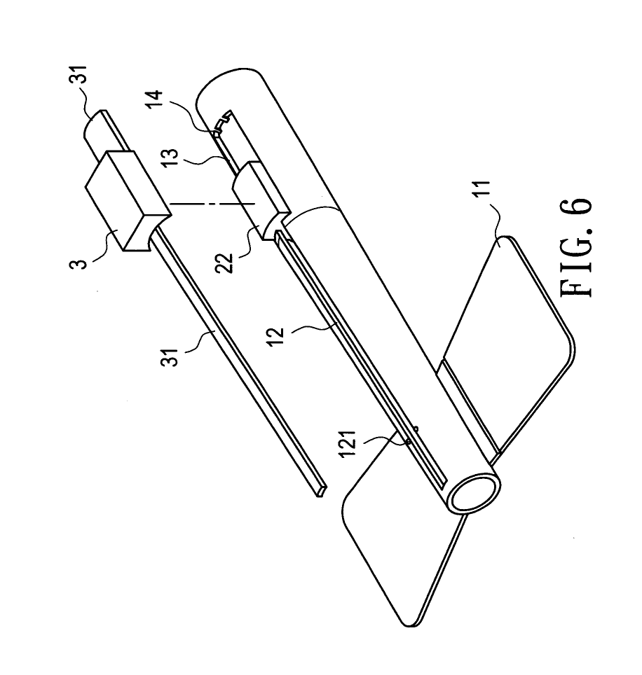

Referring to FIGS. 5, 5A and 6, in the one-time safe administration set structure according to the invention, no protruding button 121 is provided on a position near the rear end of the slideway 12, but a locating portion 14 is provided by extending from one or two sides of the protective rider 1, where on the locating portion 15, there is provided a locating slice 151 for connecting with the hand pushing part 22 through a connecting part 152. With this design, prior to use of the one-time safe administration set structure according to the invention, the gliding part 2 will be fixed and can not move forward to lead the injection needle 23 penetrating the protective rider 1 and hence avoids any risk of accidental hurt. Further, in the use of the one-time safe administration se structure according to the invention, the connecting part 152 can be broken simply by pushing the hand pushing part 22 forwardly and then the gliding part 2 can be pushed further to the front end of the slideway 12 for carrying out the operation of injection or drawing. Alternatively, in the one-time safe administration set structure according to the invention, instead of providing the locating portion 15 and the locating slice 151, the hand pushing part 22 can be connected directly with the protective rider 1 through a connecting part 122. Further, said connecting part 152 can be replaced with glue or other connecting element. Moreover, prior to the use of the one-time safe administration set structure according to the invention, the hand pushing part 22 of the gliding part 2 can be covered with a covering piece 3. A covering body 31 is provided by extending from the front end and rear end of the covering piece 3 for inserting in the slideway 12 and the receptive groove 13, respectively, to prevent the gliding part 2 from gliding.

Referring to FIG. 5B, in the one-time safe administration set structure according to the invention, a fixing slice 18 can be provided on one side of the hand pushing part 22, and at the bottom of the fixing slice 18, a groove 181 is provided, so that, as the gliding part 2 is pushed to the front edge of the protective rider 1 along the slideway 12, by bending the fixing slice 18 or the butterfly slice 11 of the protective rider 1, the butterfly slice 11 can be forced into the groove 181 so as to prevent the gliding part 2 from receding that may cause difficulty on drawing or injection.

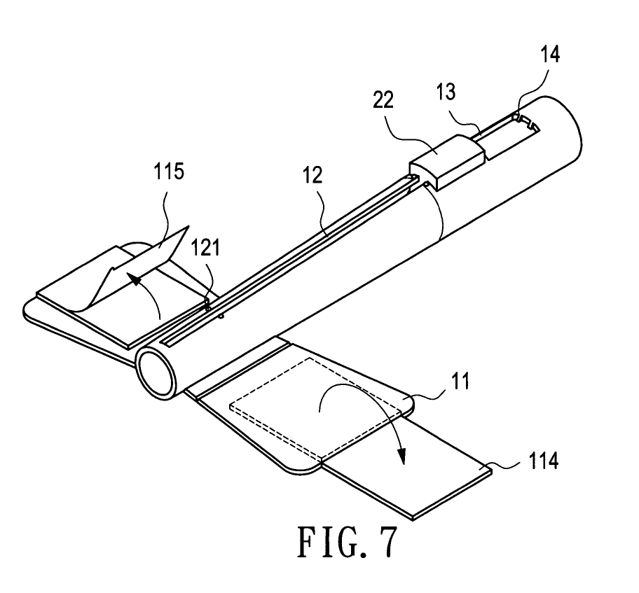

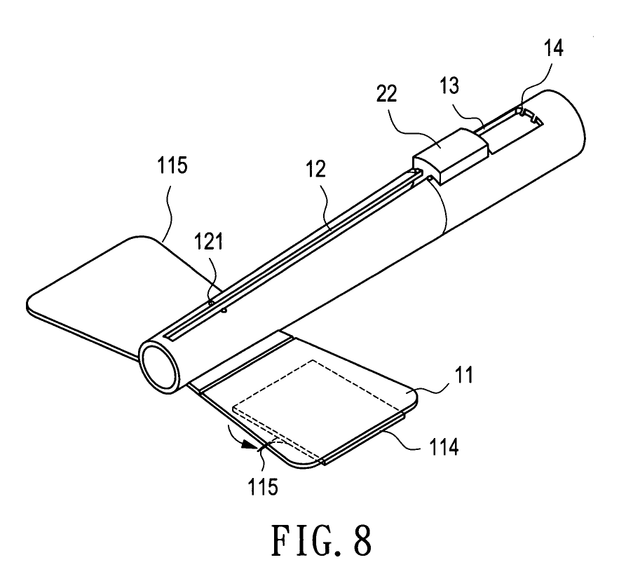

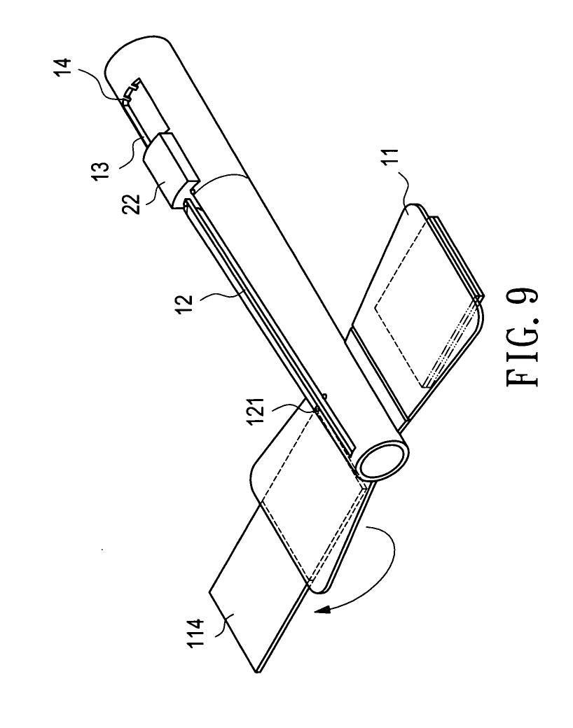

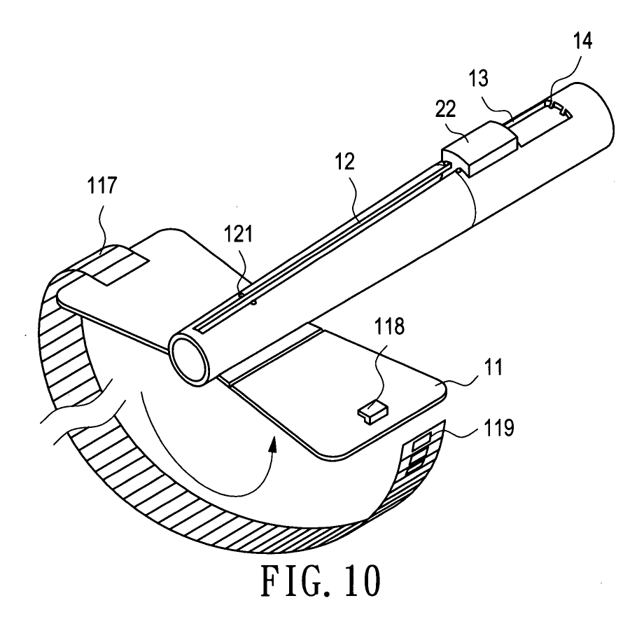

Referring to FIGS. 7, 8, 9 and 10, in the one-time safe administration set structure according to the invention, an adhesive part 114 can be attached along the side edge on the top of the butterfly slice 11 or at the bottom of the butterfly slice 114, wherein said adhesive part 114 is covered with a membrane 115. Upon inserting the injection needle 23 at the front end of the gliding part 2 into the injection site of the subject, the membrane 115 can be peeled off, and the adhesive part 114 is turned over with its junction with the butterfly slice 11 as the axis, and then sticks to the side of the injection site on the subject for locating the one-time safe administration set structure according to the invention at place during injection or drawing. Alternatively, the adhesive part 114 can be provided beneath the butterfly slice 11 in a manner that the adhesive part 114 is connected with another adhesive part 114 at its side edge. When the one-time safe administration set structure according to the invention is to be used, simply turning can do the sticking over the lower adhesive part 114 with the connection line as the axis. Furthermore, a crimping slice 117 can be provided on the butterfly slice 11 at one side of the protective rider 1, wherein several penetrating holes 119 are provided on one end of said crimping slice, and a fastening hook 118 is provided on another butterfly slice 11, such that, by bringing the crimping slice 117 over the injection site (the hand or foot) of the subject, and fastening different penetrating hole 119 into the fastening hook 118, and adjusting the distance surrounded by the crimping slice 117, the purpose of locating the one-time safe administration set structure according to the invention at place can be thus achieved. Alternatively, the fastening hook 118 and the penetrating holes 119 can be replaced by a Velcro tape.

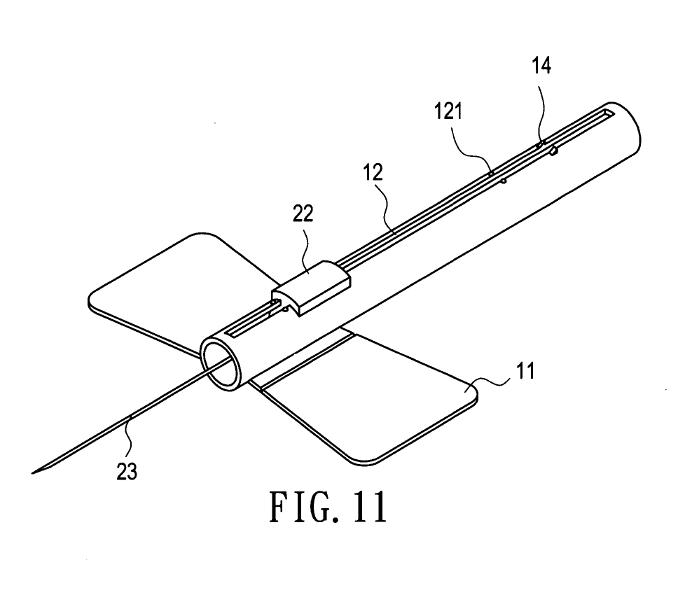

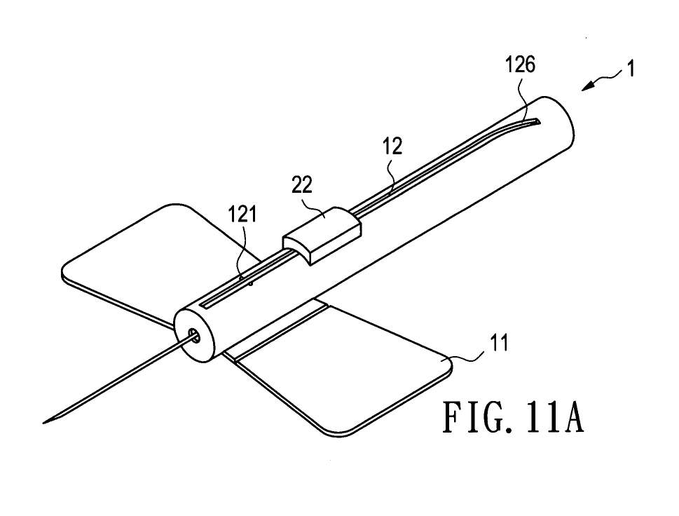

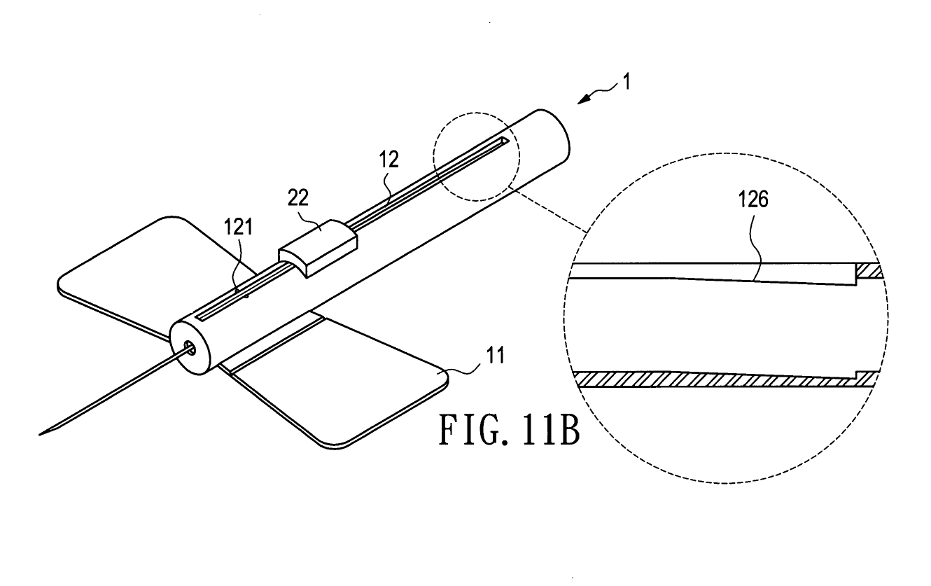

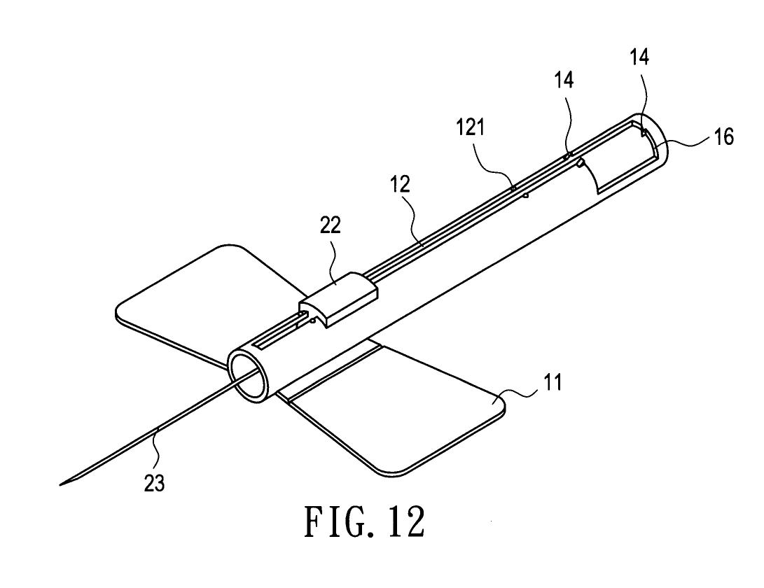

Referring to FIGS. 11 and 12, in another embodiment of the one-time safe administration set structure according to the invention, the protective rider 1 is not tapered forwardly from the protruding button 121 at the rear end of the slideway 12, and no receptive part 13 is provided at the rear end of the slideway 12 of the protective rider 1, but a stopping block 14 is provided at outside place near the end of the slideway 12, such that, as the one-time safe administration set structure according to the invention is used, by simply pushing the hand pushing part 22 backward and passing over said stopping block 14, and hence the hand pushing part 22 is resisted by the stopping block 14, the gliding part 2 will not no longer move forwardly. Further, a tucking portion 16 is provided into the inside edge near the rear end of the slideway 12 of the protective rider 1, and several stopping blocks 14 are provided at the inside edge of the tucking portion 16 with this design, as the one-time safe administration set structure according to the invention is used, by simply pushing the gliding part 2 to the rear end of the slideway 12 and placing the hand pushing part 22 into the tucking portion 16 such that the hand pushing part 22 will be resisted by the stopping block 14, and no longer turned out.

Referring to FIGS. 11A and 11B in another embodiment of the one-time safe administration set structure according to the invention, instead of providing a stopping block 14 at the rear end of the slideway 12, a deviation angle 126 is generated over the inner wall surface at the rear end of the slideway 12 or the rear end of the protective rider 1, as well as a annular stopping portion 10 is provided at the front end of the protective rider 1 for penetrating off by the injection needle 23 only. With this, when the injection or drawing by means of the one-time safe administration set structure according to the invention is complete, through pushing the gliding part 2 backward by means of the hand pushing part 22, the gliding part 2 will be affected by the deviation angle 126 and render the tip of the injection needle 23 to be tilted to the extent that it will be resisted by the annular stopping portion 10, so that the injection needle 23 can no longer to penetrate out of the protective rider 1 and hence can avoid its secondary use.

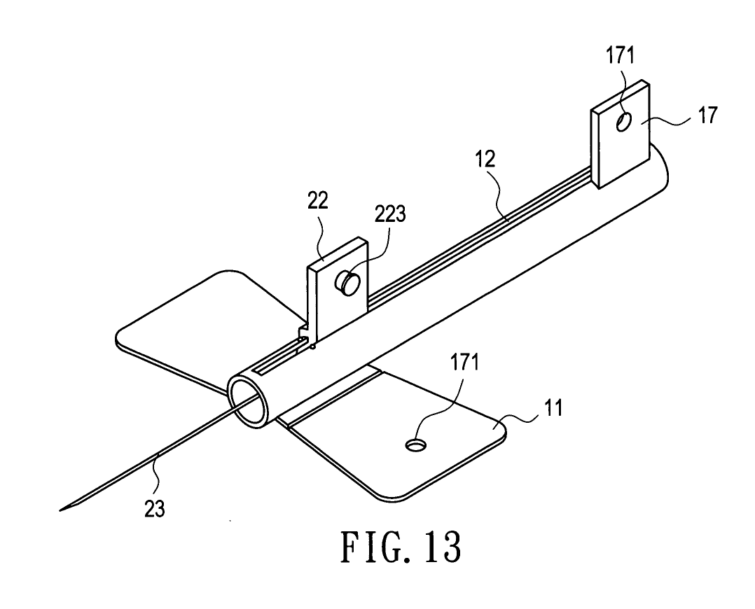

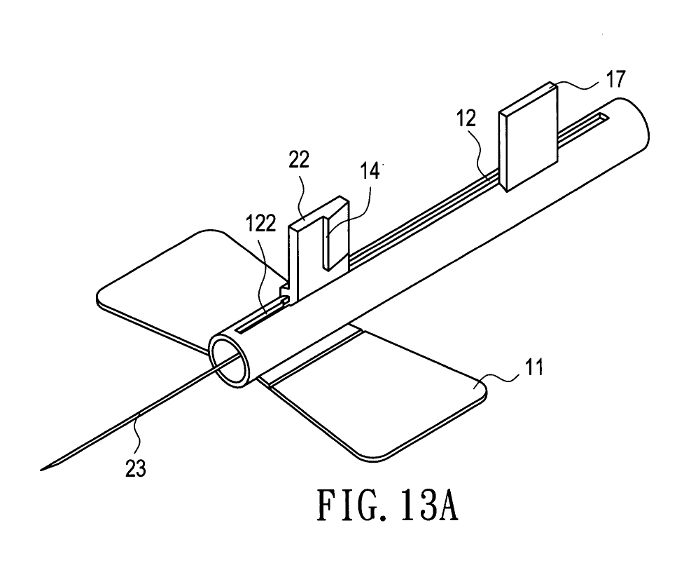

Referring to FIG. 13 and 13A, in another embodiment of the one-time safe administration set structure according to the invention, no protruding button 121, stopping block 14 and receptive portion 12 are provided at the rear end of the slideway 12, but a fixing block 17 having a fastening part 171 is extended upwardly at one side on the rear end of the slideway 12, and a fastening part 171 is provided on the hand pushing part 22 of the gliding part 2 to engage with the fastening part 171 on the fixing block 17. Alternatively, no fastening part 171 is provided on the fixing block 17, while the fastening part 223 on the hand pushing part 22 is replaced with a stopping block 14. With this design, as the use of the one-time safe administration set structure according to the invention is complete, by simply pushing the hand pushing part 22 backward to let the stopping block 14 resist against the rear wall surface of the fixing block 17, the one-time safe administration set structure according to the invention will no longer be used again.

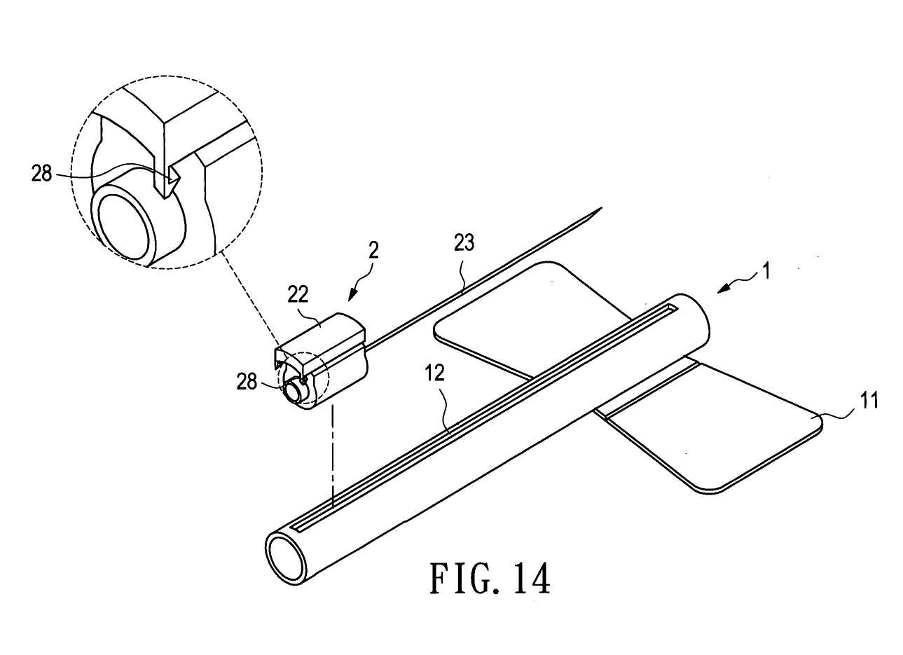

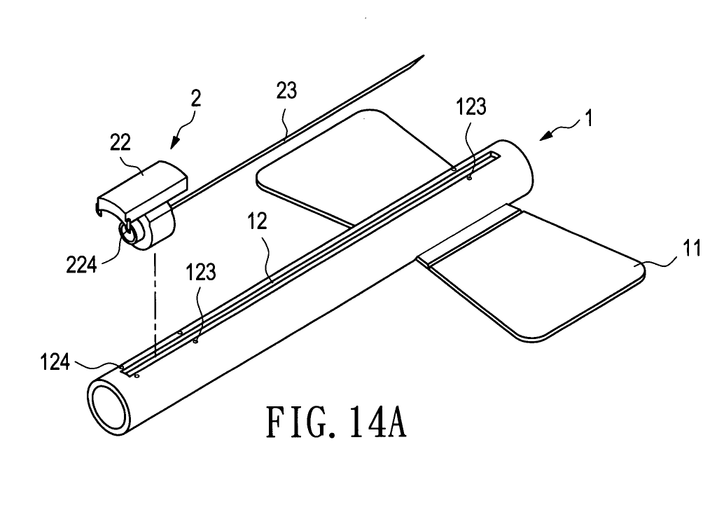

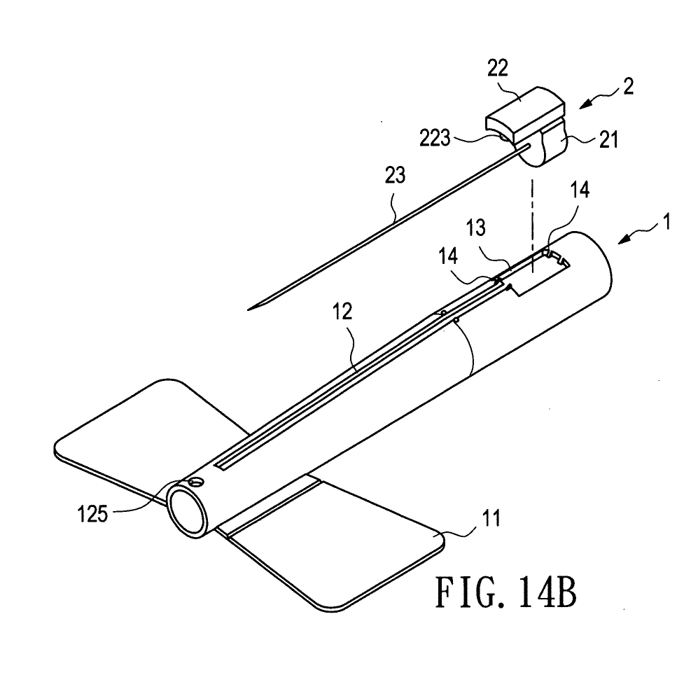

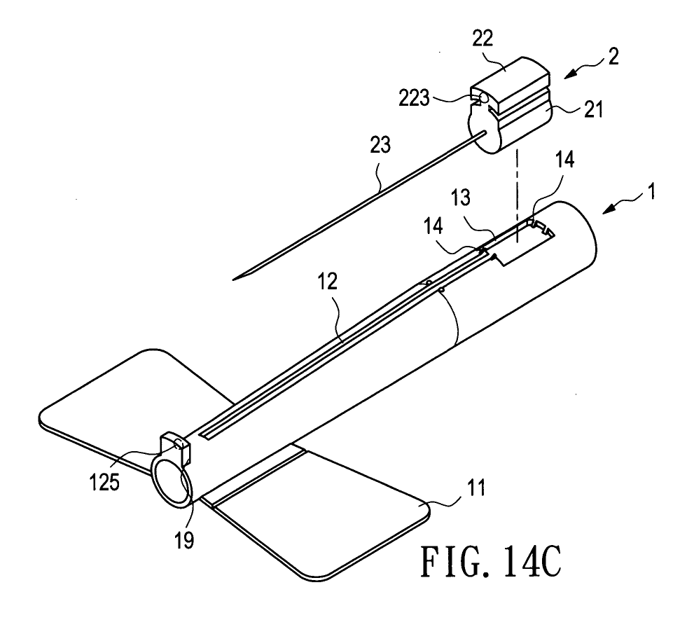

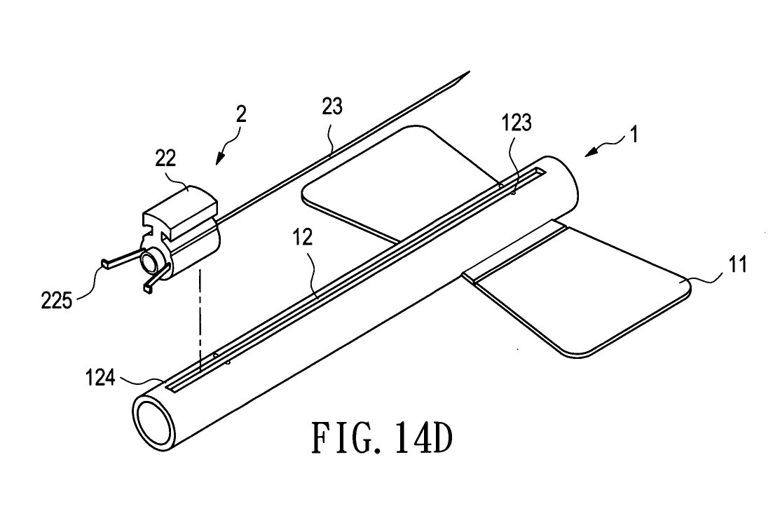

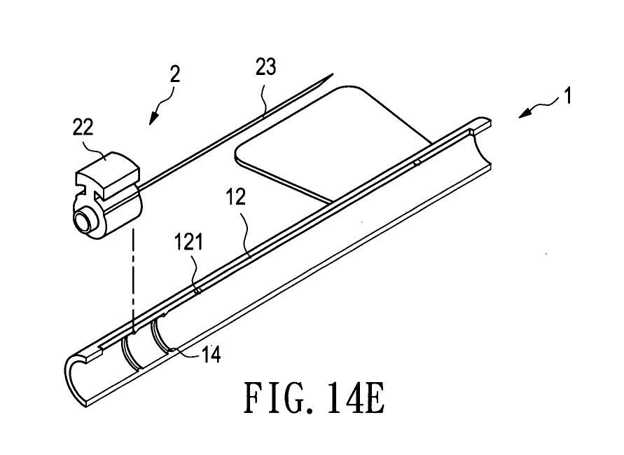

Referring to FIG. 14 and 14A, in another embodiment of the one-time safe administration set structure according to the invention, one or more fastening hook 28 is extended downwardly from the rear end of the hand pushing part 22 in a manner that the fastening hook 28 can engage with the inside edge of the protective rider 1. Thus, as the drawing or injection by using the one-time safe administration set structure according to the invention is complete, by pushing the gliding block 21 to the rear end of the protective rider 1 such that the fastening hook 28 at the rear end of the gliding block 21 engages with the inside edge of the protective rider 1, and the gliding block 21 can no longer bring the gliding part 2 moving forwardly, thereby the injection needle 23 is prevented from penetrating out of the protective rider 1 and thus avoids dangerous risk. Further, in one embodiment of the one-time safe administration set structure according to the invention, a hanging hook 224 is extended downwardly from one or two sides of the rear end of the hand pushing part 22, while several recesses 123 are provided on one or two sides of the slideway 12 of the protective rider 1, and a penetrating hole 124 is further provided on one or two sides near the end of the slideway 12 of the protective rider 1. Thus, as the one-time safe administration set structure according to the invention is used, by inserting the 54Xying hook 224 into the recess 123, the injection needle 23 will not be brought to displace by the gliding part 2 and hence avoids any difficulty during injection or drawing. Furthermore, as the use of the one-time safe administration set structure according to the invention is complete, by simply pushing the gliding part 2 to the end of the slideway 12 to let the hanging hook 224 insert into the penetrating hole 124, the gliding part 2 can be fixed at the end of the slideway 12 and hence prevent the secondary use of the one-time safe administration set structure according to the invention.

Referring to FIG. 14B and 14C, in another embodiment of the one-time safe administration set structure according to the invention, a protruding button (a post) 223 is also provided at the bottom of the front end of the hand pushing part 22, and a recess button (a socket) 125 is provided the front outer edge of the protective rider 1. With this, when the gliding part 2 is pushed to the front end of protective rider 1, by virtue of snapping the protruding button 223 into the recess button 125, the injection needle 23 can be prevent from receding. When the drawing or injection is complete, by simply pushing the gliding part 2 backward, the protruding button 223 will detach from the recess button 125. Alternatively, said protruding button 223 may be deposited at the front edge of the hand pushing part 22, while a protruding block 19 is provided at the outer front edge of said protective rider 1, and wherein a concave button 125 is provided in the protruding block 19 at a position corresponding to the protruding button 223 on the front end of the hand pushing part 22. Additionally, the protruding button 223 and the recess button 125 on the hand pushing part 2 and the protective rider 1 can be exchanged with each other.

Referring to FIG. 14D, in another embodiment of the one-time safe administration set structure according to the invention, the stopping block 14 at the rear edge of the slideway 12 of the protective rider 1 may be replaced with a protruding button 121, and additionally, one or more elastic hook 225 may be provided at the rear side of the gliding part 2. Prior to the use of the one-time safe administration set structure according to the invention, said hand pushing part 22 is restricted between two protruding button 121 at the rear side of the slideway 12 while the elastic hook 225 at the rear end of the gliding part 2 is kept against the wall surface of the inner edge of the protective rider 1. As the use of the one-time safe administration set structure according to the invention is complete, the gliding part 2 can be pushed to the rearmost end of the slideway 12 simply by means of the hand pushing part 22, and then, said elastic hook 225 will penetrate out of the opening at the rear end of the protective rider 1 and, by virtue of its elasticity, the elastic hook 225 will bounce out bilaterally such that the elastic hook 225 can hook at the rear side of the protective rider 1 to render the gliding part 2 can no longer to be pushed forward and hence achieve the purpose of preventing its secondary use.

Referring to FIG. 14E, in another embodiment of the one-time safe administration set structure according to the invention, protruding buttons 121 and stopping blocks 14 at the front and rear ends of slideway 12 can be provided, alternatively, at the inner edge of the protective rider 1. Further, one or more stopping block 14 can be provided around the inner edge of the protective rider 1. With this, as the use of the one-time safe administration set structure according to the invention is complete, the gliding part 2 can be pushed backward by means of the hand pushing part 22 where, by the voice or hand feeling generated at the time the gliding part 2 passing over the stopping block 14, it can be confirmed that the gliding part 2 has passed over and fixed at the rear side of the stopping block 14 such that the gliding part 2 can no longer be pushed forward and hence achieve the purpose of preventing its secondary use.

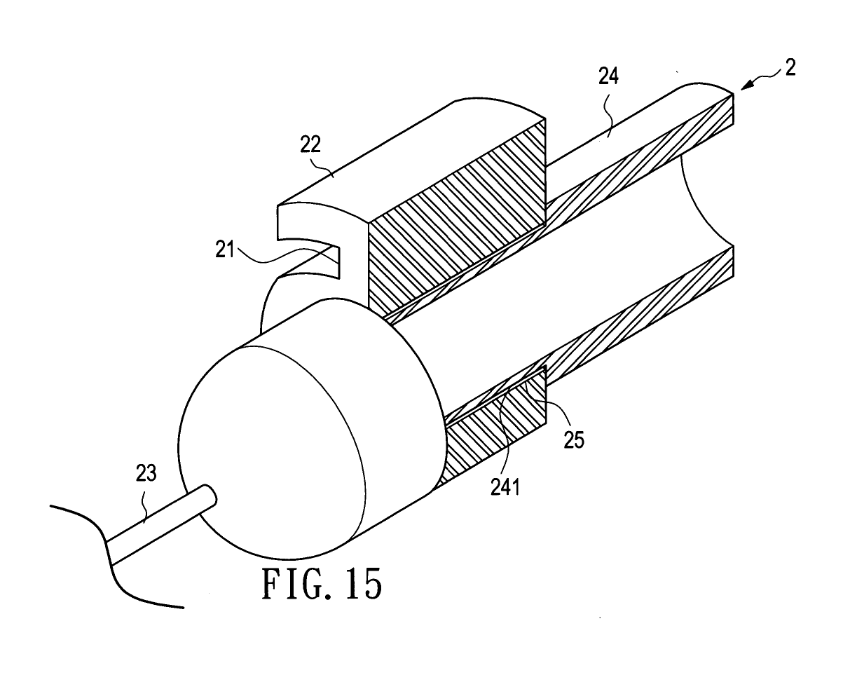

Referring to FIG. 15, in another embodiment of the one-time safe administration set structure according to the invention, the gliding part 2 is composed of a main body 24 and a hand pushing part 22, wherein said main body 24 is inserted therein an injection needle 23, while an annular recess 241 is provided inwardly, and a fastening hole 25 having a protruding rib 251 is provided at the middle of the hand pushing part 22, wherein said protruding rib 251 encloses the outer periphery of the annular recess 241. With this configuration of the one-time safe administration set structure according to the invention, the direction of the slope over the tip of the injection needle 23 can be adjusted by rotating the main body 24. Further, several marks can be provided on the outer edge of the main body 24 at places corresponding to the position of the slope over the tip of the injection needle 23 so that, when the injection needle 23 is to be inserted into the injection site of the subject and the main body is rotated, the medical practitioner is able to notice the direction of the slope over the tip of the injection needle 23.

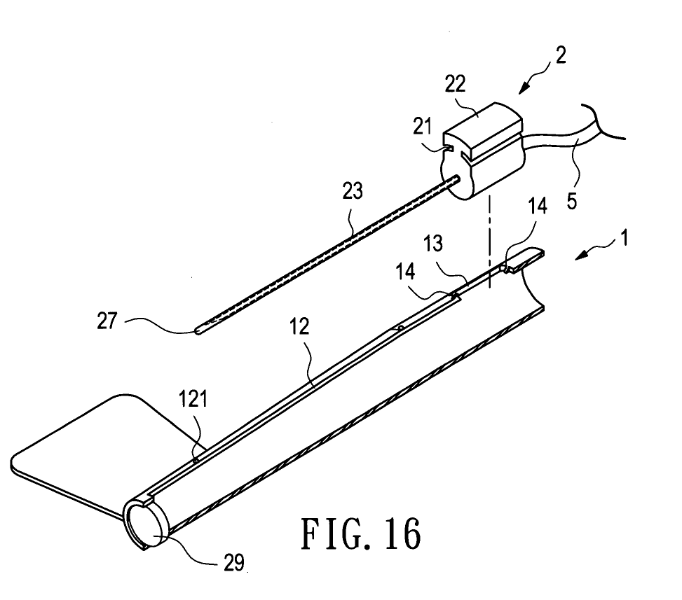

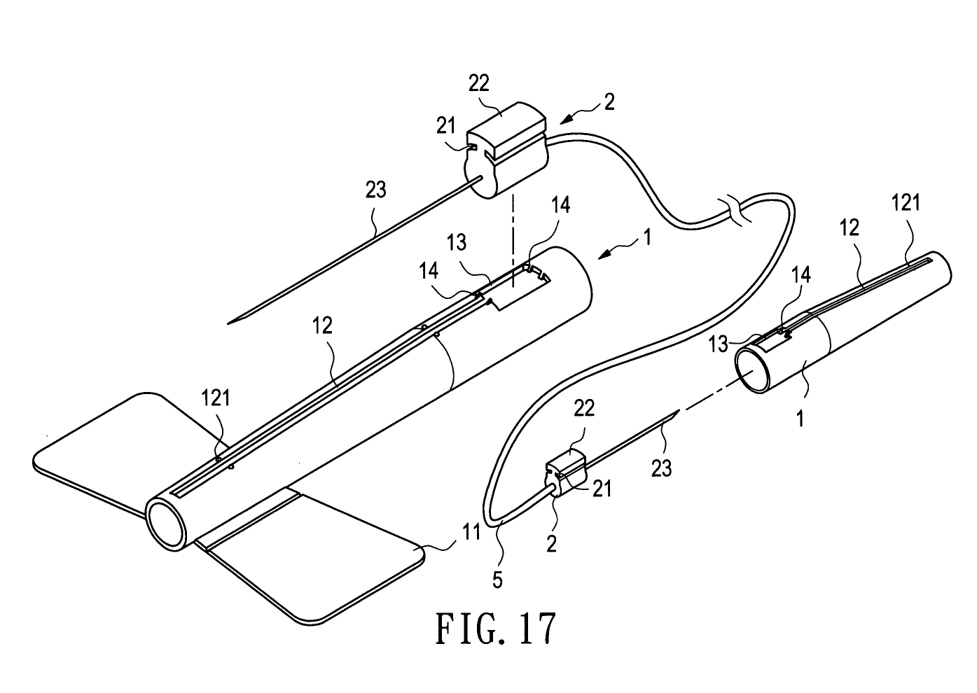

Referring to FIGS. 16 and 17, in another embodiment of the one-time safe administration set structure according to the invention, the adapting head 51 connecting with the rear end of the gliding part 2 through a hose 5 is replaced with an injection needle 23, where said injection needle 23 is inserted at the rear side of the gliding part 2 and said gliding part 2 is provided with a gliding block 21 to connect with a hand pushing part 22. Furthermore, the gliding part 2 is set within a protective rider 1, wherein said protective rider 1 is provided with a slideway 12 that is provided at its rear side with a receptive portion 13, while a protruding button 121 is provided near the front and rear ends of the slideway 12, respectively, and, besides, several stopping blocks 14 are provided at the junction between the slideway 12 and the receptive portion 13 as well along the periphery of the receptive portion 13. Moreover, said protective rider 1 is tapered forwardly from the protruding button 121 at the rear end of the slideway 12. In addition, the gliding part 2 and the protective rider 1 just mentioned can be the same as shown in FIGS. 1A, 1B, 1F, 5, 5A, 6, 11, 11A, 11B, 12, 13, 13A, 14, 14A, 14B, 14C, 14D, 14E and 15, and no butterfly slice 11, fastening slice and handling portion are provided at both sides of the protective rider 1, too. Further, in one embodiment of the one-time safe administration set structure according to the invention, an outer annular jacket 27 made of an elastic, flexible rubber material is provided around the outer periphery of the injection needle 23. When the one-time safe administration set structure according to the invention is used for injecting or drawing and inserting the injection needle 23 into the injection site of a subject or into a test tube, the outer annular jacket 27 will be receded due to the resisting from the skin or the test tube, while the injection needle penetrates the injection site of the subject or into the test tube. After the drawing or injection is finished, as the injection needle 23 is withdrawn from the injection site, the outer annular jacket will enclose the outer periphery of the injection needle 23 again due to its flexible property, and the site of the outer annular jacket 27 penetrated by the injection needle 23 will be forced to be sealed closely due to the elasticity of the outer annular jacket 27, such that, the risk of infection by the blood remained on the injection needle 23 can be avoided. In additional, the function of the outer annular jacket 27 can be replaced by providing an elastic rubber material 29 over the front opening of the protective rider 1.

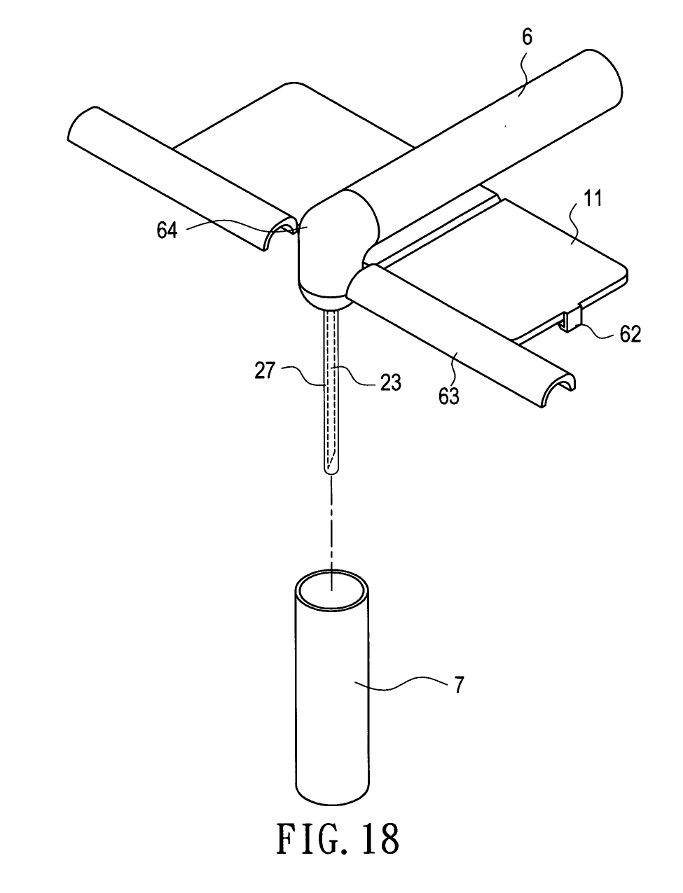

Referring to FIG. 18, the one-time safe administration set structure according to the invention comprises:

an administration set main body 6, its front end bending downwardly and being provided with an injection needle 23 inserted therein, wherein a butterfly slice 11 is extended outwardly from the lower edge of both sides of said administration set main body 6, and wherein the butterfly slice 11 on one side is provided on the inner edge with one or more engaging hook 62, and a semi-arc cover 63 is provided on front ends of both butterfly slices 11, while the rear end of the administration set main body 6 is connected with a hose 5; and

a needle thimble 7, sleeving on the injection needle 23 of the administration set main body 6.

When the one-time safe administration set structure according to this embodiment of the invention is used to carrying out the operation of drawing or injection, the needle thimble 7 on the administration set main body is simply removed, and the butterfly slices 11 are bended over, such that the medical practitioner can insert the injection needle 23 into the injection site of a subject to carry out the operation of drawing or injection. Thereafter, it is possible to avoid the risk of exposing the injection needle by simply bending downwardly the butterfly slices 11 on both sides, engaging one butterfly slice 11 on one side with the butterfly slice 11 on another side with the engaging hook 62, and, due to the engagement of butterfly slices 11 on both sides, semi-arc covers 63 at ends of the two butterfly slices 11 will cover together the injection needle 23, so as to avoid the occurrence of danger caused by exposure of the injection needle 23. Alternatively, no engaging hook 62 is provided on the butterfly slice 11 on one side of the administration set main body 6, but fasteners that can fastened with each other are provided at the bottom of two butterfly slices 11 or at the junction between two semi-arc covers 63. Furthermore, the structure according to the invention can be attached with the rear end of the hose 5 for connecting a test tube. Moreover, an outer annular jacket 27 made of elastic, flexible rubber material is provided to enclose the outer edge of the injection needle 23, such that, when the operation of drawing is carried out, the administration set in front of the hose 5 can be used to insert the injection site of a subject (such as, for drawing blood, ascitic fluid or other sampling work), whereupon the injection needle 23 of the structure of the invention is inserted into a test tube, and at this time, the outer annular jacket 27 will recede due to resistance from the test tube, and thereby lead the penetration of the injection needle into the test tube. As the drawing is complete, the medical practitioner can bend butterfly slice 11 on both sides downwardly and pulls correspondingly the injection needle 23 out of the test tube, such that, butterfly slices 11 on both sides can be fastened with each other through the engaging hook 62 and semi-arc cover 63 at the front end of the two butterfly slices 11 can enclose the injection needle 23, thereby danger from exposure of the injection needle can thus be avoided. Furthermore, the site on the outer annular jacket 27 punctured by the injection needle 23 can be forced to be seal closely by virtue of the elasticity of the outer annular jacket 27 so that the risk of infection by the exposure in the air of the blood remained on the injection needle can be avoided simultaneously.

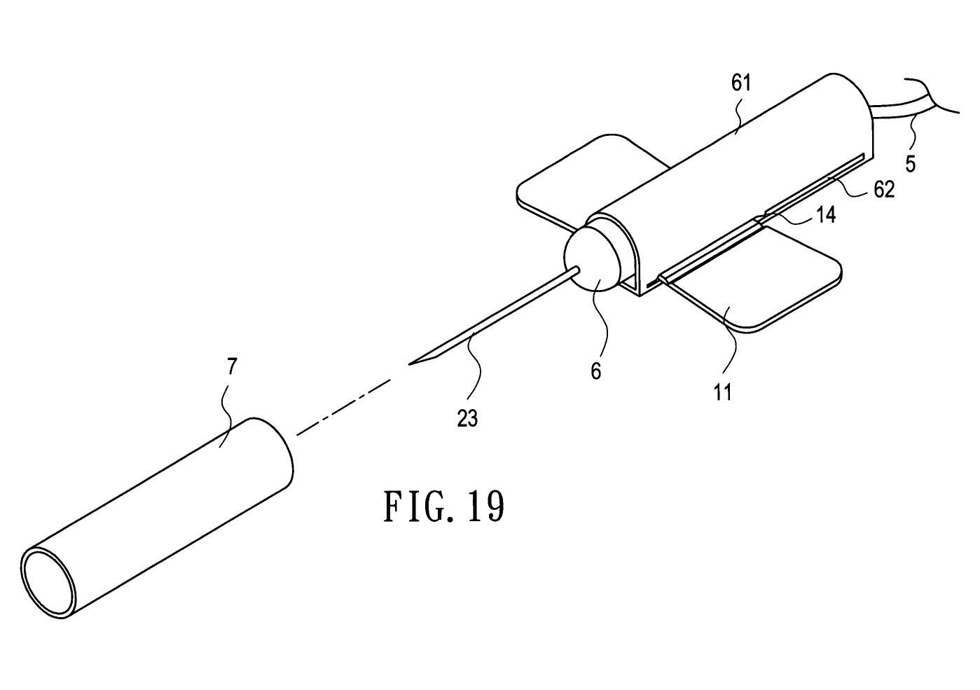

Referring to FIG. 19, the one-time safe administration set structure according to the invention comprises:

an administration set main body 6, being provided with an injection needle 23 inserted therein, wherein a butterfly slice 11 is extended outwardly from the lower edge of both sides of said administration set main body 6, and wherein the rear end of the administration set main body 6 is connected with a hose 5;

a needle thimble 7, sleeving on the injection needle 23 of the administration set main body 6; and

a gaiter 61, sleeving on said administration set main body 6, wherein a slot 62 for the penetrating of the butterfly slice 11 is provided at the side edge of said gaiter 61, and wherein the front edge of said slot 62 is in a design of tapering with a stopping block 14 provided at a position near the rear side of said slot 62.

With the forgoing, before the use of the one-time safe administration set structure provided by the invention, the gaiter 61 presses against the butterfly slice 23 of the administration set main body 6 through a tapered design of its front opening such that the gaiter 61 can not move arbitrarily. When the one-time safe administration set structure according to the invention is used, the needle thimble is removed from the administration set main body 6 and then the operation of drawing or injection is carried out. After the operation is complete, the gaiter is pushed forwardly to let the butterfly slice 11 pass over the stopping block 14 at the opening 62 of the gaiter 61 so as to prevent the one-time safe administration set structure according to the invention form secondary use.

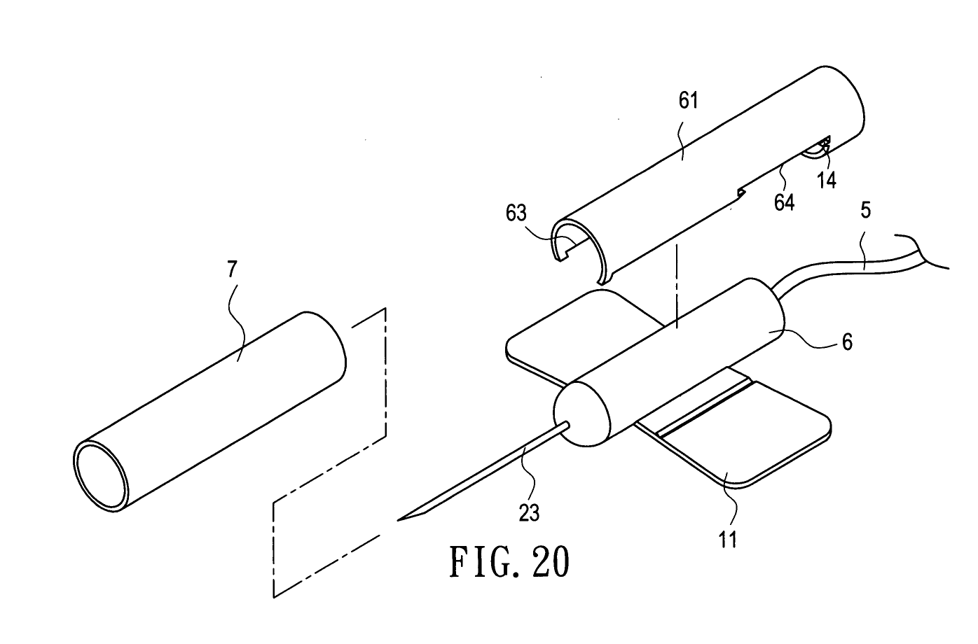

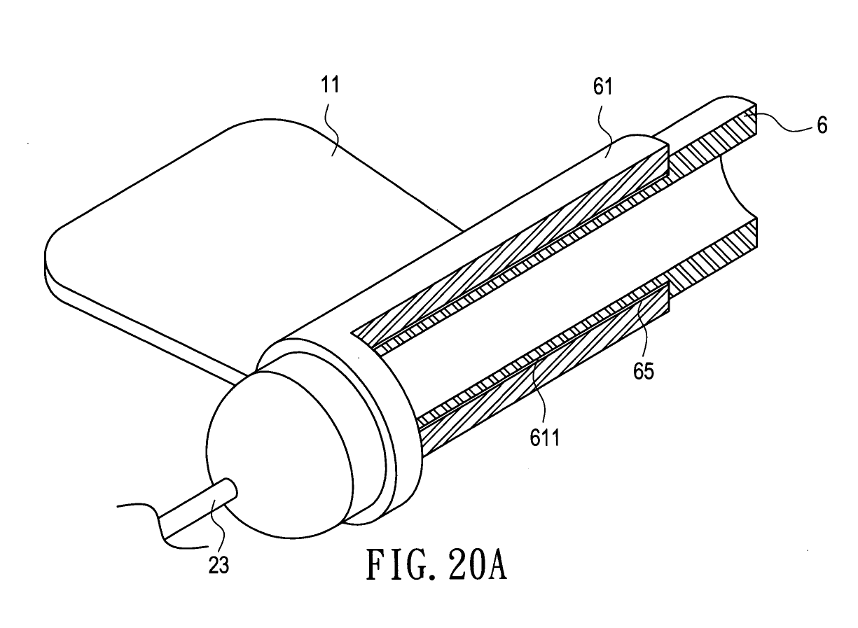

Referring to FIG. 20, in one embodiment of the one-time safe administration set structure according to the invention, the design of the bottom of said gaiter 61 is a sliding portion 63 that recesses upwardly. The sliding portion 63 has, at its rear side, a receptive portion 64 that recesses upwardly. Further, several stopping block are provided at the junction between the sliding portion 63 and the receptive portion 64. With this design, as the use of the one-time safe administration set structure according to the invention is complete, by simply pushing the gaiter 61 to the front edge of the administration set main body 6 and then pressing the gaiter downwardly, or bending the butterfly slice 11 upwardly, the butterfly slice 11 will be placed in the receptive portion 64, and can not release from the receptive portion due to the resisting of the stopping block 14, thereby it is possible to prevent the one-time safe administration set structure according to the invention from secondary use.

Referring to FIG. 21A, in one embodiment of the one-time safe administration set structure provided by the invention, an annular recess slot 65 is provided inwardly at a middle part of the administration set main body 6, while a gaiter 61 encloses the outer periphery of the administration set main body 6. A butterfly slice 11 is provided extending outwardly from the lower edge of one or two sides of said gaiter 61, respectively. A protruding rib 611 is provided on the inner edge of the gaiter 61 at a position corresponding to the annular recess slot 65. With this design of the one-time safe administration set structure provided by the invention, the direction of the slope over the tip of the injection needle 23 can be adjusted by the administration set main body 6 or the gaiter 61. Alternatively, several marks can be provided on the outer edge of the administration set main body 6 at places corresponding to the position of the slope over the tip of the injection needle 23 so that, when the injection needle 23 is to be inserted into the injection site of the subject and the main body 6 is rotated, the medical practitioner is able to notice the direction of the slope over the tip of the injection needle 23.

The one-time safe administration set structure provided by the invention has been described with reference to embodiments thereof. It should be noted that the one-time safe administration set structure provided by the invention can be applied to various syringes, such as, for example, specific syringe for hemodialysis, blood sampler, common needle type syringe or various adapting tube such as, transfusion tube.

Many changes and modifications in the above-described embodiment of the invention can, of course, be carried out without departing from the scope thereof. Accordingly, to promote the progress in science and the useful arts, the invention is disclosed and is intended to be limited only by the scope of the appended claims.

What is claimed is:

1. A one-time safe administration set, comprising:
a protective rider with a front end and a rear end provided therein with one or more butterfly slice extended laterally from the front, bottom of said protective rider, and also provided with a slideway, where said slideway is provided on its rear side with a receptive portion, and with one or more protruding buttons near its front and rear ends, and wherein said protective rider is provided with several stopping blocks at a junction between said slideway and said receptive portion as well as around said receptive portion; and further, said protective rider is tapered forwardly from the protruding button at its rear end;
a gliding part, engaged with a hand pushing part through a sliding block on an upper edge of said gliding part;
a hose, provided at its end with an adapting head which can connect with a usual syringe for injection, or drawing, or catheter for infusion;
wherein said gliding part is set by inserting in said protective rider, and is fixed at a place between said protruding button at the rear end of said slideway and said stopping block.

2. A one-time safe administration set as recited in claim 1, wherein said adapting head can be replaced with a screw adapting head that can connect with a special syringe having a screw thread.

3. A one-time safe administration set as recited in claim 1, wherein, at the rear end of said hose, it being possible to connect with a syringe which can be provided with an injection needle and wherein an external annular jacket is provided around the periphery of said injection needle.

4. A one-time safe administration set as recited in claim 1, wherein said protective rider may not take a form of being tapered forwardly from the protruding button provided at the rear end of the slideway, but instead said protective rider can be raised upwardly from the protruding button provided at the rear end of the slideway.

5. A one-time safe administration set as recited in claim 1, wherein the protruding button on the protective rider and the stopping block provided at the junction between the slideway and the receptive portion can be provided around the outside periphery of the protective rider, and at a junction with the bottom of the hand pushing part.

6. A one-time safe administration set as recited in claim 1, wherein said protective rider may not be necessarily provided with said stopping block and the protruding button, and said protective rider may not take a form of being tapered forwardly from said protruding button provided at the rear end of the slideway, and instead, a protruding block is provided at the end tail part of said the hand pushing part of the gliding part.

7. A one-time safe administration set as recited in claim 1, wherein a protruding portion is provided at the bottom of the front end of the hand pushing part and wherein the front and rear ends of said protruding portion has a slope.

8. A one-time safe administration set as recited in claim 1, wherein said protruding button can be provided at the front edge of said hand pushing part, and wherein a protruding block is provided at the front end of the outer edge of the protective rider, and wherein a concave button is provided on said protruding block at a position corresponding to said protruding button at the hand pushing part, or, alternatively, said protruding button on the hand pushing part and the concave button on said protruding block can be provided interchangeably.

9. A one-time safe administration set as recited in claim 1, wherein said stopping block at the rear edge of said slideway of the protective rider can be replaced with a protruding button, and wherein one or more elastic hook is provided at the rear side of said gliding part.

10. A one-time safe administration set as recited in claim 1, wherein the front end of said slideway can be in an invaginate form, while said slideway is not provided with any protruding block at a position near its front end.

11. A one-time safe administration set as recited in claim 1, wherein an opening is further provided on the bottom at the front of said protective rider.

12. A one-time safe administration set as recited in claim 1, wherein a butterfly slice is provided on both sides of said protective rider, and no protruding button is provided at a position near the front end of said slideway, while a stopping block is provided on the butterfly slice at one or two sides of said protective rider.

13. A one-time safe administration set as recited in claim 1, wherein a butterfly slice is provided on both sides of said protective rider, and no protruding button is provided at a position near the front end of said slideway, while a protruding portion is provided extending outwardly from one or two sides of said hand pushing part, respectively, and a grommet for fastening to the corresponding protruding portion is provided on said butterfly slice of said protective rider.

14. A one-time safe administration set as recited in claim 1, wherein a butterfly slice is provided on both sides of said protective rider, and no protruding button is provided at a position near the front end of said slideway, while a recess is provided on one or two sides of said hand pushing part and a fastening block is provided on said butterfly slice of said protective rider for fastening into the recess.

15. A one-time safe administration set as recited in claim 1, wherein no protruding button is provided at a position near the front end of said slideway, while a locating portion is provided by extending from one or two sides of said protective rider, wherein on the locating portion, there is provided a locating slice for connecting with said hand pushing part through a connecting part.

16. A one-time safe administration set as recited in claim 1, wherein no locating portion and locating slice is provided, and said hand pushing part is connected directly with the protective rider through a connecting part, and wherein said connecting part is replaced with a glue or other connecting element.

17. A one-time safe administration set as recited in claim 1, wherein a fixing slice is provided on one side of said hand pushing part, and a groove is provided at the bottom of said fixing slice.

18. A one-time safe administration set as recited in claim 1, wherein no protruding button is provided at a position near the front end of said slideway, while said hand pushing part of said gliding part is covered with a covering piece and a covering body is provided by extending from the front end and rear end of said covering piece for inserting in the slideway and the receptive groove, respectively.

19. A one-time safe administration set as recited in claim 1, wherein an adhesive part is attached along the side edge on the top of said butterfly slice, and wherein said adhesive part is covered with a membrane.

20. A one-time safe administration set as recited in claim 19, wherein said adhesive part is attached at the bottom of said butterfly slice, and wherein said adhesive part is covered with a membrane.

21. A one-time safe administration set as recited in claim 1, wherein an adhesive part is provided beneath the butterfly slice in a manner that said adhesive part is connected with another adhesive part at its side edge.

22. A one-time safe administration set as recited in claim 1, wherein a butterfly slice is provided on both sides of said protective rider, wherein a crimping slice is provided on the butterfly slice at one side of said protective rider, and wherein several penetrating holes are provided on one end of said crimping slice, and a fastening hook is provided on another butterfly slice.

23. A one-time safe administration set as recited in claim 22, wherein said fastening hook and said penetrating holes are replaced by a Velcro tape.

24. A one-time safe administration set as recited in claim 1, wherein said protective rider is not tapered forwardly from the protruding button at the rear end of the slideway, and no receptive part is provided at the rear end of said slideway of said protective rider, and wherein a stopping block is provided at an outside place near the end of said slideway.

25. A one-time safe administration set as recited in claim 24, wherein said stopping block is provided at the outer periphery of said protective rider and at the junction between said protective rider and said hand pushing part.

26. A one-time safe administration set as recited in claim 24, wherein said protruding button and stopping block at the front and rear ends of said slideway can be provided alternatively at the inner edge of said protective rider, and wherein one or more said stopping block can be provided around the inner edge of said protective rider.

27. A one-time safe administration set as recited in claim 1, wherein, instead of providing said stopping block on the rear end of said slideway, a deviation angle is generated over the inner wall surface at the rear end of said slideway or the protective rider, and wherein an annular stopping portion is further provided at the front end of the protective rider for the injection needle to penetrate out.

28. A one-time safe administration set as recited in claim 1, wherein said protective rider is not tapered forwardly from said protruding button at the rear end of said slideway, and no receptive part is provided at the rear end of said slideway of said protective rider, and wherein a tucking portion is provided into the inside edge near the rear end of said slideway of said protective rider, and several stopping blocks are provided at the inside edge of said tucking portion.

29. A one-time safe administration set as recited in claim 1, wherein said protective rider is not tapered forwardly from said protruding button at the rear end of said slideway, and none of said protruding button, said stopping block and said receptive portion is provided at the rear end of said slideway, and wherein a fixing block having a fastening part is extended upwardly at one side on the rear end of said slideway, and a fastening part is provided on said hand pushing part of said gliding part to engage with said fastening part on said fixing block.

30. A one-time safe administration set as recited in claim 29, wherein no fastening part is provided on said fixing block, and wherein said fastening part on the hand pushing part is replaced with a stopping block.

31. A one-time safe administration set as recited in claim 1, wherein no receptive part is provided at the rear end of said slideway, and said protective rider is not tapered forwardly from said protruding button at the rear end of said slideway, and wherein a fastening hook is extended downwardly from the rear end of said hand pushing part in a manner that the fastening hook can engage with the inside edge of said protective rider.

32. A one-time safe administration set as recited in claim 1, wherein a protruding button is provided at the bottom of the front end of said hand pushing part, and a recess button is provided at the front outer edge of said protective rider, and wherein said protruding button and recess button at said hand pushing part and said protective rider can be exchanged with each other.

33. A one-time safe administration set as recited in claim 1, wherein said protective rider is not tapered forwardly from said protruding button at the rear end of said slideway, and wherein a fastening hook is extended downwardly from one or two sides of the rear end of said hand pushing part and wherein several recesses are provided on one or two sides of said slideway of said protective rider, and a penetrating hole is further provided on one or two sides near the end of said slideway of said protective rider.

34. A one-time safe administration set as recited in claim 1, wherein the gliding part is composed of a main body and a hand pushing part, wherein an injection needle is provided by inserting in the front end of said main body, wherein an annular recess is provided inwardly at the middle site of said main body, and a fastening hole having a protruding rib is provided at the middle of said hand pushing part, and wherein said protruding rib encloses the outer periphery of said annular recess.

35. A one-time safe administration set as recited in claim 1, wherein said adapting head is replaced with an injection needle, where said injection needle is inserted at the rear side of said gliding part and said gliding part is provided with a gliding block to connect with a hand pushing part, and wherein said gliding part is set within said protective rider, and said protective rider is provided with a slideway that is provided at its rear side with a receptive portion, while a protruding button is provided near the front and rear ends of said slideway, respectively, and wherein several stopping blocks are provided at the junction between said slideway and said receptive portion as well as along the periphery of said receptive portion and wherein said protective rider is tapered forwardly from the protruding button at the rear end of said slideway.

36. A one-time safe administration set as recited in claim 35, wherein the front end of said slideway is tapered, and no protruding button is provided near the front end of said slideway.

37. A one-time safe administration set as recited in claim 35, wherein no protruding button is provided near the front end of said slideway, and wherein said hand pushing part of said gliding part is covered with a covering piece, and wherein a covering body is provided by extending from the front end and rear end of said covering piece for inserting in said slideway and said receptive groove, respectively.

38. A one-time safe administration set as recited in claim 35, wherein said protective rider is not tapered forwardly from the protruding button provided at the rear end of said slideway, and no receptive part is provided at the rear end of said slideway, and wherein said protective rider is raised upwardly from said protruding button provided at the rear end of said slideway.

39. A one-time safe administration set as recited in claim 35, wherein said protective rider is not tapered forwardly from the protruding button provided at the rear end of said slideway, and no receptive part is provided at the rear end of said slideway, wherein said protruding button and stopping block at the front and rear ends of said slideway can be provided alternatively at the inner edge of said protective rider, and wherein one or more said stopping block can be provided around the inner edge of said protective rider.

40. A one-time safe administration set as recited in claim 35, wherein said protective rider is not tapered forwardly from the protruding button provided at the rear end of said slideway, and no receptive part is provided at the rear end of said slideway, and wherein a tucking portion is provided into the inside edge near the rear end of said slideway of said protective rider, and several stopping blocks are provided at the inside edge of said tucking portion.

41. A one-time safe administration set as recited in claim 35, wherein said protective rider is not tapered forwardly from said protruding button at the rear end of said slideway, and no stopping block is provided at the rear end of the slideway, while a deviation angle is generated over the inner wall surface at the rear end of said slideway or said protective rider and an annular stopping portion is also provided at the front end of said protective rider for the injection needle only to penetrate out.

42. A one-time safe administration set as recited in claim 35, wherein said protective rider is not tapered forwardly from said protruding button at the rear end of said slideway, and none of said protruding button, said stopping block and said receptive portion is provided at the rear end of said slideway, and wherein a fixing block having a fastening part is extended upwardly at one side on the rear end of said slideway, and a fastening part is provided on said hand pushing part of said gliding part to engage with said fastening part on said fixing block.

43. A one-time safe administration set as recited in claim 42, wherein said fastening part on the hand pushing part is replaced with a stopping block.

44. A one-time safe administration set as recited in claim 35, wherein an outer annular jacket made of an elastic, flexible rubber material is provided around the outer periphery of the injection needle or over the front opening of said protective rider.

45. A one-time safe administration set as recited in claim 35, wherein neither said stopping block nor said protruding button is provided on said protective rider and said protective rider is not tapered forwardly from said protruding button at the rear end of said slideway, and wherein a fastening hook is extended downwardly from the rear end of said hand pushing part.

46. A one-time safe administration set as recited in claim 35, wherein a protruding button is provided at the bottom of the front end of said hand pushing part, and a recess button is provided at the front outer edge of said protective rider, and wherein said protruding button and recess button at said hand pushing part and said protective rider can be exchanged with each other.

47. A one-time safe administration set as recited in claim 35, wherein neither said stopping block nor said protruding button is provided on said protective rider and said protective rider is not tapered forwardly from said protruding button at the rear end of said slideway, and wherein a protruding block is provided at the bottom of said hand pushing part of said gliding part.

48. A one-time safe administration set as recited in claim 35, wherein a protruding portion is provided at the bottom of the front end of the hand pushing part, and wherein the front and rear ends of said protruding portion has a slope.

49. A one-time safe administration set as recited in claim 35, wherein said protruding button is provided at the front edge of said hand pushing part, and a protruding block is provided at the front outer edge of said protective rider, and wherein a concave button is provided on said protruding block at a position corresponding to said protruding button at front end of the hand pushing part, or, alternatively, said protruding button on the hand pushing part and the concave button on said protruding block can be provided exchangeably.

50. A one-time safe administration set as recited in claim 35, wherein a said stopping block at the rear edge of said slideway of the protective rider can be replaced with a protruding button, and wherein one or more elastic hook is provided at the rear side of said gliding part.

51. A one-time safe administration set as recited in claim 35, wherein said protective rider is not tapered forwardly from said protruding button at the rear end of said slideway, and wherein a fastening hook is extended downwardly from one or two sides of the rear end of said hand pushing part, several recesses are provided on one or two sides of said slideway of said protective rider, and a penetrating hole is further provided on one or two sides near the end of said slideway of said protective rider.

52. A one-time safe administration set as recited in claim 35, wherein no protruding button is provided at a position near the front end of said slideway, while a locating portion is provided by extending from one or two sides of said protective rider, wherein on the locating portion, there is provided a locating slice for connecting with said hand pushing part through a connecting part.

53. A one-time safe administration set as recited in claim 52, wherein no locating portion and locating slice is provided, and said hand pushing part is connected directly with said protective rider through a connecting part, and wherein said connecting part is replaced with a glue or other connecting element.

54. A one-time safe administration set as recited in claim 35, wherein the gliding part is composed of a main body and a hand pushing part, wherein an injection needle is provided by inserting in the front end of said main body, and an annular recess is provided inwardly at the middle site of said main body, and a fastening hole having a protruding rib is provided at the middle of said hand pushing part, and wherein said protruding rib encloses the outer periphery of said annular recess.

55. A one-time safe administration set as recited in claim 1, wherein said adapting head connected with the rear end of said hose is replaced with an administration set, wherein the front end of said administration set bends downwardly and is provided with an injection needle inserted therein, and wherein an outer annular jacket made of an elastic, flexible rubber material is provided around the outer periphery of the injection needle, and wherein a butterfly slice is provided by extending outwardly from the lower edge of both sides of said administration set main body, and the butterfly slice on one side is provided on the inner edge with one or more engaging hook, and wherein a needle thimble is provided sleeving on front end of said administration set.

56. A one-time safe administration set as recited in claim 55, wherein no engaging hook is provided on said butterfly slice at one side of the administration set main body, wherein fasteners that can fastened with each other or glue are provided at the bottom of two butterfly slices or at the junction between two semi-arc covers.

57. A one-time safe administration set structure comprising:
an administration set main body, being provided with an injection needle inserted therein, wherein an outer annular jacket made of an elastic, flexible rubber material is provided around the outer periphery of the injection needle, and wherein a butterfly slice is extended outwardly from the lower edge of both sides of said administration set main body, and wherein, the rear end of the administration set main body is connected with a hose;
a needle thimble, sleeving on said injection needle of said administration set main body; and
a gaiter, sleeving on said administration set main body, wherein a slot for the penetrating of the butterfly slice is provided at the side edge of said gaiter, and wherein the front edge of said slot is in a design of tapering with a stopping block provided at a position near the rear side of said slot;
wherein said gaiter presses against said butterfly slice of said administration set main body through a tapered design of its front opening.

58. A one-time safe administration set as recited in claim 57, wherein the bottom of said gaiter is provided with a sliding portion that recesses upwardly, and wherein said sliding portion is provided, at its rear side, with a receptive portion that recesses upwardly, and several stopping block are provided at the junction between said sliding portion and said receptive portion.

59. A one-time safe administration set as recited in claim 57, wherein said hose is connected with an adapting head, and wherein said adapting head is replaced with a screw adapting head that can screw engaged with a special syringe having screw thread.

60. A one-time safe administration set as recited in claim 57, wherein a syringe is connected with the rear end of said hose, and wherein an injection needle is provided in said syringe, said injection needle is enclosed by a outer annular jacket.

61. A one-time safe administration set as recited in claim 57, wherein an adhesive part is attached along the side edge on the top of said butterfly slice, and wherein said adhesive part is covered with a membrane.

62. A one-time safe administration set as recited in claim 61, wherein said adhesive part is provided at the bottom of said butterfly slice, and wherein said adhesive part is covered with a membrane.

63. A one-time safe administration set as recited in claim 57, wherein said adhesive part is provided beneath said butterfly slice in a manner that said adhesive part is connected with another adhesive part at its side edge.

64. A one-time safe administration set as recited in claim 57, wherein butterfly slices are provided by extending from both sides of said protective rider, and wherein a crimping slice is provided on said butterfly slice at one side of said protective rider, and wherein several penetrating holes are provided on one end of said crimping slice, and a fastening hook is provided on another butterfly slice.

65. A one-time safe administration set as recited in claim 64, wherein said fastening hook and said penetrating hole is replaced with a Velcro tape.

66. A one-time safe administration set as recited in claim 57, wherein an administration set is connected with the rear end of said hose, wherein the front end of said administration set bends downwardly and is provided with an injection needle inserted therein, and wherein an outer annular jacket made of an elastic, flexible rubber material is provided around the outer periphery of the injection needle, and wherein a butterfly slice is provided by extending outwardly from the lower edge of both sides of said administration set, and the butterfly slice on one side is provided on the inner edge with one or more engaging hook, and wherein a needle thimble is provided sleeving on front end of said administration set.

67. A one-time safe administration set as recited in claim 66, wherein no engaging hook is provided on said butterfly slice at one side of the administration set main body, wherein fasteners that can fastened with each other or glue are provided at the bottom of two butterfly slices or at the junction between two semi-arc covers.

68. A one-time safe administration set as recited in claim 57, wherein an annular recess slot is provided inwardly at a middle part of the administration set main body, and wherein a gaiter encloses the outer periphery of the administration set main body; wherein a butterfly slice is provided extending outwardly from the lower edge of one or two sides of said gaiter, respectively; and wherein a protruding rib is provided on the inner edge of said gaiter at a position corresponding to said annular recess slot.

69. A one-time safe administration set, comprising:
an administration set main body, its front end bending downwardly and being provided with an injection needle inserted therein, wherein a butterfly slice is extended outwardly from the lower edge of both sides of said administration set main body, and wherein said butterfly slice on one side is provided on the inner edge with one or more engaging hook, and a semi-arc cover 63 is provided on front ends of both butterfly slices, and wherein the rear end of said administration set main body is connected with a hose; and
a needle thimble, sleeving on the injection needle of said administration set main body.

70. A one-time safe administration set as recited in claim 69, wherein no engaging hook is provided on said butterfly slice at one side of the administration set main body, and wherein fasteners that can fastened with each other or glue are provided at the bottom of two butterfly slices or at the junction between two semi-arc covers.

71. A one-time safe administration set as recited in claim 69, wherein said hose is connected with an adapting head, and wherein said adapting head is replaced with a screw adapting head that can screw engaged with a special syringe having screw thread.

72. A one-time safe administration set as recited in claim 69, wherein a syringe is connected with the rear end of said hose, and wherein an injection needle is provided in said syringe, said injection needle is enclosed by an outer annular jacket.

73. A one-time safe administration set as recited in claim 69, wherein an adhesive part is attached along the side edge on the top of said butterfly slice, and wherein said adhesive part is covered with a membrane.

74. A one-time safe administration set as recited in claim 73, wherein said adhesive part is provided at the bottom of said butterfly slice, and wherein said adhesive part is covered with a membrane.

75. A one-time safe administration set as recited in claim 69, wherein said adhesive part is provided beneath said butterfly slice in a manner that said adhesive part is connected with another adhesive part at its side edge.

76. A one-time safe administration set as recited in claim 69, wherein butterfly slices are provided by extending from both sides of said protective rider, and wherein a crimping slice is provided on said butterfly slice at one side of said protective rider, and wherein several penetrating holes are provided on one end of said crimping slice, and a fastening hook is provided on another butterfly slice.

77. A one-time safe administration set as recited in claim 76, wherein said fastening hook and said penetrating hole is replaced with a Velcro tape.
